(19) United States
(12) Patent Application Publication
(10) Pub. No.: US 2008/0107652 A1
(43) Pub. Date: May 8, 2008

(54) Methods and compositions for control of disease in aquaculture

(75) Inventors: Ravi Durvasula, Albuquerque, NM (US); Subba Durvasula, Darthmouth (CA)

Correspondence Address: LAW OFFICES OF KHALILIAN SIRA, LLC, 9100 PERSIMMON TREE ROAD, POTOMAC, MD, 20854, US

(73) Assignee: SCIENCE & TECHNOLOGY CORPORATION @ UNIVERSITY OF NEW MEXICO STC.UNM

(21) Appl. No.: 11/893,699

(22) Filed: Aug. 17, 2007

Related U.S. Application Data
(60) Provisional application No. 60/840,278, filed on Aug. 25, 2006.

(51) Int. Cl. A61K 35/66; A61K 39/00; A61K 39/395; A61K 31/70; A61P 43/00

(52) U.S. Cl. 424/135.1; 424/93.2; 424/184.1; 514/44; 424/164.1; 424/159.1

(57) ABSTRACT

The invention discloses paratransgenesis methods for prevention, amelioration or treatment of a disease or disorder in an aquatic animal. The method comprises providing a genetically modified micro algae that expresses a recombinant molecule that specifically targets one or more key epitopes of a pathogen that infects the aquatic animal and ii) feeding the aquatic animal directly or indirectly with the genetically modified unicellular algae.

### CROSS REFERENCE TO RELATED APPLICATIONS

This application claims benefit of priority to the provisional patent application No. 60/840,278, filed on Aug. 25, 2006, the entire contents of which are incorporated herein by reference.

### GOVERNMENT FUNDING

The present invention was made in part with salary support from the Veteran's Administration.

### I. FIELD OF THE INVENTION

The invention relates to methods and compositions for the control of infections in aquaculture. In particular, the invention relates to methods for the control of infections in commercial aquaculture by paratransgenesis.

### II. BACKGROUND OF THE INVENTION

World aquaculture production has increased to 59.4 million metric tons (MT) in 2004, with a value of $70 billion. Of this, farmed shrimp production accounts for 2.4 million MT, representing a value of nearly $10 billion (FAO 2004). Diseases caused by agents such as White Spot Syndrome Virus (WSSV) and Vibrio species have decimated shrimp farming industries in many parts of Asia and South America, and account for nearly $3 billion of economic loss annually. Unregulated use of antibiotics in farmed shrimp and fish operations has widely been banned and contributes to the epidemic of drug-resistant bacteria in humans. Intensive practices that involve meticulous water exchange with strict standards of hygiene have been effective in reducing transmission of infectious pathogens in farmed shrimp (Otoshi et al. 2001 and 2002), but are impractical in many lower-income settings of the world. Usually, appearance of disease is associated with loss of harvest for shrimp farmers which accounts for the loss of 30% of global production. The economic impact of infectious diseases of mariculture is overshadowed only by their tremendous threat to global food security.

The United States is the second largest importer of shrimp in the world. Shrimp aquaculture, like other animal husbandry industries, is subject to disease, especially under current intensive farming methods. In the USA, more than 50 diseases are associated with aquaculture operations and affect shellfish and fish. For example in the shrimp industry, diseases are associated with parasites (70%), bacteria (27%) and fungi (3%) caused by about 20 pathogens including the gastero-intestinal Vibrio harveyi, V parahaemolyticus and V vulnificus. It is of interest to note a) of the nine known pathogenic strains of Vibrio, five are common to humans, and b) natural assemblages of algae live in association with several species of bacteria and viruses.

To prevent the diseases, it is crucial to understand the functioning of the pathogens, how they affect the commercially important high-density stressed mariculture operations, and how the marine animal would fight the disease. The rapid growth of this industry has outpaced efforts by researchers, pharmaceutical companies, and federal regulatory agencies to provide approved therapeutics for disease management of marine. Currently, there are no antibacterials approved for shrimp aquaculture in the U.S. Oxytetracycline (OTC) and Romet-30 are two antibacterials currently approved in the U.S. for catfish and salmonid aquaculture. Several combative methods based on drugs are administered. Included in this are naturotherapy (latex from Swallowwort, neemcake), chemotherapy (chlorine, ozone, iodine and formalin). Antibiotics such as Chlorapmphenicol, Oxytetracycline, Tetracycline, Ampicillin, Bacitracin, Gentamycin, Neomycin, Streptomycin, Penicillin G, Polymixin-B and Sulphadiazine are routinely used at shrimp aquaculture facilities outside of the U.S. (Park et al, 1994). These have limited success due to evolution of pathogen resistance to antibiotics.

Biotechnological approaches hold a promise in the prevention, control and management of disease and disorders associated with marine culture. One approach is to use genetically transformed strains of a mariculture that are resistant to pathogen invasion. For example, procedures for germ line transformation of shrimp have been successfully established at UMBI, Center of Marine Biotechnology, Baltimore, Md., in France at IFREMER and in Australia at CSIRO. Here the researches have worked on introducing DNA into shrimp by transfection and followed expression and integration of the introduced DNA in the host. These procedures can be exploited to produce pathogen-resistant shrimp.

Whereas germline transformation of mariculture holds promise, issues remain regarding the role of genetically modified organisms as human food. Furthermore, stability of germline transformation and viability of genetically modified offspring may present challenges.

The application of transgenic technologies to marine and freshwater algae, diatoms and cyanobacteria is a new and rapidly evolving field. Whereas the genetic composition of some of these organisms is well characterized, application of recombinant DNA technologies to generate biologically enhanced or augmented forms is at a nascent stage. The expression of foreign, biologically active molecules by genetically modified algae offers great potential for large-scale and economical production of many proteins of commercial and therapeutic significance.

Several reports indicate that algae such as Chlamydomonas reinhardtii (Mayfield 2003) and Phaeodactylum tricornutum (Zaslavskaia 2001) may be genetically manipulated to express heterologous proteins. Using a chloroplast transformation system, Mayfield et al. demonstrated expression of a functional large single-chain (lsc) antibody in C. reinhardtii. The antibody, directed against glycoprotein D of human herpes simplex virus, was produced in solubilized form by the alga and assembled into higher order complexes in vivo. In an earlier study, Zaslavskaia et al. engineered P. tricornutum with either a human (glut 1) or Chlorella (hup 1) glucose transporter gene. The resulting conversion of a photosynthetic autotroph to a heterotroph capable of obtaining exogenous glucose in the absence of light energy was a significant advance in algal biotechnology.

Of the total 40,000 species of micro algae, 4500 are marine species of which 250 are known to grow rapidly leading to either seasonal or atypical bloom formation. A study of the blooms is important not only for their contribution to trophodynamics of the ecosystem but also due to mass mortalities of several biota associated with anoxic conditions resulting from disintegrating organic mass. There are approximately hundred micro-algae that produce specific toxins (Fogg 2002). Of these, about 60 dinoflagellates are known to cause red tides and some produce toxins causing Diaetic Shellfish Poisoning (DSP), Paralytic Shellfish Poisoning (PSP), Neurotoxin poisoning (NSP) and Ciguatera. Because some of these algae are consumed either as food or passively filtered and retained by the commercially important shellfish, bioaccumulation of toxins takes place in the marine food web.

Red tide organisms are known to cause severe economic losses and set backs to human health. Globally the economic losses could be as high as US$ 20 billion and 3.5 to 7 million disability adjusted life-years, much in excess of those caused by Chagas disease (GESAMP 2001) and are comparable to those caused by epidemics such as malaria, and diabetes. Additionally, bacterial and viral contamination of the water may cause considerable mortality to larvae of commercially important species.

Marine diatoms have also been investigated for genetic studies. Only a few marine diatoms Skeletonema costatum, Cyclotella cryptica, Navicula saprophila and Phaeodactylum tricornutum are so far utilized in gene transformation studies. Smith and Alberte (1995) have succeeded in transferring the animal virus SV40 or plant virus CaMV35S promoters into a marine diatom Skeletonema costatum. Although no stable integration was achieved, β-glucoronidase and luciferase have been expressed in S. costatum reporter genes. More recently, using particle bombardment technique on diatom cultures, where a high pressure helium pulse delivers nucleic acids, Dunahay et al (1995) in Cyclotella cryptica, Navicula saprophila and Apt et al (1996) and Zaslavskaia et al (2000) in Phaeodactylum tricornutum succeeded stable transformation of DNA. Further, Zaslavskaia et al (2001) have successfully genetically engineered and converted a photosynthetic diatom Phaeodatylum tricornutum to grow on exogenous glucose in the dark.

The bottleneck for genetic transformation of diatoms was resolved in 1995. Dunahay et al generated lines of transgenic Cyclotella cryptica and Navicula saprophila with plasmid vectors containing the E. coli neomycin phosphtransferase II gene using helium accelerated particle bombardment (Dunahay et al. 1995). This was followed by the successful transformation of Phaeodactylum tricornutum (Apt et al. 1996) and Cylindrotheca fusiformis (Fisher et al. 1999). A landmark transformation study was demonstrated by Zaslavskaia et al in 200 r. Most diatoms are solely photosynthetic and lack the ability to grow in the absence of light. These investigators successfully engineered P. tricornutum, a photosynthetic diatom, to grow on exogenous glucose in the dark by transformation with the glucose transporter gene Glut1 from human erythrocytes or Hup1 from the microalga Chlorella kessleri. Positive transformants exhibited glucose uptake and grew in the dark in the presence of glucose (Zaslavskaia et al. 2001). The exciting trophic conversion of an obligate photoautotrophic diatom is a critical first step for successful large-scale cultivation using microbial fermentation technology. Commercial benefits from such a system are enormous, ranging from an increase in biomass and productivity to reduced loss from contamination by obligate phototropic microbes.

The multicellular organism Volvox carteri represents an ideal model organism to study the transition from unicellularity to multicellularity. Using C. reinhardtii as a model, stable nuclear transformation of V. carteri was reported in 1994 by Schiedlmeier et al. Elegant studies with the intent of generating selectable markers for gene replacement and gene disruption analysis were subsequently developed (Hallmann and Sumper 1994). One of these studies resulted in a V. carteri transformant that carried the Chlorella hexose/H⁺ symporter that is able to survive in the presence of glucose in the dark (Hallmann and Sumper 1996). As in the case with diatoms, this development will only accelerate the development of commercial expression systems for V. carteri.

In all these investigations the most important objective was the stable integration of the transgenes, their autonomous replication and proper expression of the gene product. It is imperative that in mariculture operations the nutritional content of the genetically modified alga is not significantly altered and the product is quite similar to that of a non-genetically modified organism.

Marine Cyanobacteria or the blue greens are ubiquitous and in the open ocean account for 50% of photosynthetic production (Platt, Subba Rao and Irwin 1983). Although Cyanobacteria are more exacting in their growth requirements, they are amenable to culture under laboratory conditions. Besides feeding commercially important animals such as shellfish and larvae, these cultures find applications in natural products such as pigments, pharmaceuticals, fatty acids, polysaccharides, wastewater treatment, and biodegradation of pollutants (Elhai 1994).

The green alga Chlamydomonas reinhardtii has long served as a model system for photosynthesis and flagellar. This unicellular green alga will grow on a simple medium of inorganic salts in the light, using a photosynthesis system that is similar to that of higher plants to provide energy. Chlamydomonas will also grow in total darkness if an alternate carbon source, usually in the form of acetate, is provided. Both the ˜15.8 Kb mitochondrial genome (Genbank accession: NC001638 (Vahrenholz et al. 1993)) and the complete >200 Kb chloroplast genome for this organism are available online (Genbank accession: BK000554 (Maul et al. 2002)). The current assembly of the nuclear genome is available online at http://genomejgi-psf.org/Chlre3/Chlre3.info.html. The Chlamydomonas Center located at www.chlamy.org continues to be an informative resource to the Chlamydomonas community.

Dunaliella is a unicellular, bi-flagellated green alga that belongs to the class Chlorophyceae. Morphologically, Dunaliella is very similar to Chlamydomonas. Both organisms have complex life cycles that encompass, in addition to division of motile vegetative cells, the possibility of sexual reproduction. These organisms are both photosynthetic, and relatively easy to maintain in a laboratory setting. Unlike Chlamydomonas, the genetics of Dunaliella are poorly understood. Dunaliella is by far one of the most salt-tolerant eukaryotic organisms (Ben-Amotz and Avron 1990). Furthermore, it is highly resistant to stresses such as high light intensity and dramatic pH and temperature changes. Although there is an increasing interest in the mechanisms that allow such physiological versatility, research in this area is still in its infancy. To date, few of these stressed-induced genes have been cloned from Dunaliella (Fisher et al. 1996; Fisher et al. 1997; Sanchez-Estudillo et al. 2006), and the information that is available has shed little light on the genomic organization or the biological significance of some of the unique sequence features that have been identified (Sun et al. 2006a).

One of the stress-induced responses in Dunaliella is the production and accumulation of the carotenoid, 0-carotene. Dunaliella is one of the richest natural producers of carotenoid, producing up to 15% of its dry weight under suitable conditions. Interestingly, it is thought that the carotenoid functions as a “sun-screen” to protect chlorophyll and DNA from harmful UV-irradiation (Ben-Amotz et al. 1989). The commercial cultivation of Dunaliella began in the 1960's once it was realized that their halotolerance allowed for monoculture in large brine ponds. The ease of maintaining Dunaliella in culture, its ability to grow in very high salt concentrations, tolerance to high temperature and to extreme pH changes, makes this species a highly desirable target for exploitation as a biological factory for the large-scale production of foreign proteins.

Although the genetics of Dunaliella are poorly understood, this organism is highly suited as an algal bioreactor. It can be cultured easily, rapidly and inexpensively. Until recently, the use of Dunaliella was limited by the absence of an efficient and stable transformation system. The first report of successful manipulation of D. salina was by Geng et al. in 2003. Using electroporation, these investigators were able to generate stable transformants carrying the hepatitis B surface antigen. Walker et al. in 2005 reported the isolation and characterization of two D. tertiolecta nuclear RbcS genes and their corresponding 5′ and 3′ regulatory sequences. The functionality of these regulatory regions was initially used to drive the expression of a selectable marker in C. reinhardtii. Subsequently, this expression cassette was electroporated into Dunaliella where both stable and transient transformants expressing the ble resistance gene were isolated. Jiang et al. (2005) identified and later used the 5′ flanking region of an actin gene from D. salina to direct stable expression of the bialaphos resistance gene (bar) in D. salina. In more recent work, Sun et al. (2006b) introduced a functional nitrate reductase gene into a D. salina mutant that lacked the gene. This group showed that the introduced gene was able to complement the nitrate reductase defective mutant of D. viridis. All the studies described are pivotal to the development of an effective transformation system in Dunaliella, opening the door for the use of this alga as a bioreactor for production of recombinant proteins.

In recent years, vaccines based on recombinant DNA technology appear to be a promising approach to controlling infectious diseases in farmed fish (Biering et al. 2005; Clark and Cassidy-Hanley 2005; Heppel et al. 1998). By intramuscular injection of eukaryotic expression vectors encoding the sequence of a pathogen antigen, DNA vaccines offer a method of immunization that overcomes many of the disadvantages such as risk of infection and high costs of traditional live attenuates, killed or subunit protein-based counterparts. They induce strong and long-lasting humoral and cell mediated immune responses which have made them attractive for the aquaculture industry (Heppel and Davis 2000). DNA vaccination has already been proven to be effective in rainbow trout for infectious haematopoietic necrosis virus (Boudinot et al. 1998; Corbeil et al. 1999; Kim et al. 2000; Kurath et al. 2006; Lorenzen et al. 2001; Lorenzen et al. 1999) and viral haemorrhagic septicemia virus (Lorenzen et al. 2002) as well as channel catfish for herpes virus 1 (Nusbaum et al. 2002). After intramuscular injection of plasmid DNA carrying promoter-driven reporter genes, protein expression has been achieved in common carp (Hansen et al. 1991), tilapia (Rahman and Maclean 1992), goldfish (Kanellos et al. 1999), zebrafish (Heppel et al. 1998), Japanese flounder (Takano et al. 2004) and gilthead seabream (Verri et al. 2003).

Although there are several ways to administer vaccines, most young fish continue to be vaccinated by hand. In Norway, for example, over 200 million fish are vaccinated each year. Each fish is removed from the water, anesthetized and vaccinated. This method is highly stressful for the fish, and in some circumstances rather impractical. Another method of vaccination is by dip immersion into a solution containing the vaccine. Dip immersion is usually used in fish stocks that are too young or small for manual handling. Unfortunately, this method alone is not sufficient to achieve a long duration of protection. Thus, the fish are usually subjected to intraperitoneal re-vaccination injection as soon as their size allows. Oral vaccine delivery systems are by far the most desirable method for immunizing fish. But reports have indicated that this system is ineffective. All these hurdles point to the need for the development of a more user-friendly methodology for vaccine administration (Lin et al. 2005).

White Spot Syndrome Virus (WSSV)

WSSV is the most striking example of shrimp viral disease. This disease has devastated many parts of the world with grave economic consequences and reduction in available food supply. Infection of peneaid shrimp by WSSV can result in up to 100% mortality within 3 to 7 days. The virus is extremely virulent and has a broad host range including other marine invertebrates such as crayfish and crab. The global annual economic loss due to WSSV is estimated to be $3 billion (Hill 2005). In much of the world, there is currently no effective method to control this disease.

Entry and pathogenesis of WSSV in peneaid shrimp occur either via oral ingestion or water-borne contact (Chou et al. 1998). Work by several investigators has demonstrated that VP28, a structural protein found on the virion envelope, is responsible for viral attachment, penetration and consequently the systemic infection of shrimp (Qhappel et al. 2004; van Hulten et al. 2001). Although studies on the shrimp immune response are limited, the presence of viral inhibiting proteins in both experimental and natural survivors of WSSV infections suggests that an adaptive immune response exists (Venegas et al. 2000; Wu et al. 2002). Several approaches using VP28 and another structural envelope protein, VP19, have been used to elicit an immune response in shrimp. Witteveldt et al. (2004) orally vaccinated P. monodon and L. vannamei (Witteveldt et al. 2006), two of the most important cultured shrimp species, using feed pellets coated with inactivated bacteria that were over-expressing VP28. In both cases, lower mortality was found in test versus control animals up to three weeks post vaccination. In a similar study, crayfish were protected fully from WSSV following injection with fusion VP19+VP28 polyclonal antiserum (Li et al. 2005). Vaccination trials with VP292, a newly identified envelope protein, also resulted in significant resistance to WSSV for up to 30 days post initial vaccination (Vaseeharan et al. 2006). Using a different strategy, Robalino et al. (2004, 2005) and Tirosophon et al. (2005) demonstrated that the administration of dsRNA specific for WSSV genes induces a potent and virus-specific antiviral response in shrimp. Both studies revealed significant reduction in mortality in the shrimp population protected by vp28 and vp19 dsRNA injections.

These approaches to controlling WSSV involve induction of an immune response to virulence epitopes of WSSV and suggest that this could potentially control this disease. In each approach, however, vaccine delivery constrains implementation. The method used in the studies cited above, individual inoculation of shrimp, is highly impractical under field conditions. Given that a typical shrimp grow-out pond can harbor upwards of 300,000 post-larvae per hectare, labor costs imposed by this method rule out commercial application. The coating of dry feed with inoculum appears logical, but the feeding behavior of shrimp involves the slow nibbling of feed particles. This behavior will cause substantial losses of inoculum through leaching. It has been demonstrated that within an hour, shrimp feed can lose more than 20% of its crude protein, about 50% of its carbohydrates and 85 to 95% of its vitamin content (Rosenberry 2005). In light of the tremendous global impact of WSSV on shrimp farming and the necessity of high-intensity cultivation, new strategies to impart immunity against WSSV are essential. It is also critical that such a technology be economically viable, scalable to large shrimp farming facilities, and easily delivered to the shrimp.

U.S. Patent application No. 20030211089 discloses delivery systems and methods for delivering a biologically active protein to a host animal. The systems and methods provided include obtaining an algal cell transformed by an expression vector. The biologically active protein is an antigen that upon administration to the animal induces a general immune response in the host animal.

U.S. Patent application No. 20040081638 discloses delivery of disease control in aquaculture and agriculture using nutritional feeds containing bioactive proteins produced by viruses. The gene encoding a protein or antibody is incorporated into a virus, which in turn, infects an insect organism that is a component of the feed. The virus can infect the macroalgal, plant, or animal feed component.

The invention, as disclosed and described herein, overcomes the prior art problems by providing novel approaches of paratransgenesis for transferring immunogenic peptides and antibody fragments that targets specifically one or more key epitopes of a pathogen that infects an aquatic animal.

### III. SUMMARY OF THE INVENTION

The invention provides methods of paratransgenesis for the prevention, amelioration or treatment of a disease or disorder in an aquatic animal comprising: i) providing a genetically modified microorganism that expresses one or more recombinant molecules that specifically target one or more key epitopes of a pathogen that infects an aquatic animal and ii) feeding the aquatic animal directly or indirectly with the genetically modified micro algae.

In one embodiment, the pathogen specifically infects the aquatic animal.

In another embodiment, the microorganism comprises algae, bacteria, or a combination thereof. In one embodiment, the microorganism is a micro alga, macro alga, unicellular algae, multicellular algae, or a combination thereof. In one embodiment, the microorganism is a cynobacteria, Dunaliella or a variant thereof.

In the present invention, the microorganism is transformed with a genetic material the expression products of which is one or more recombinant molecules comprising one or more antiviral or antibacterial molecules, immunogenic peptides, single chain antibody fragments, or a combination thereof. The recombinant molecules also comprise cecropins, penaeidins, bactenecins, calinectins, myticins, tachyplesins, clavanins, misgurins, pleurocidins, paras ins, hi stones, acid proteins, and lysozymes, or a combination thereof, among others.

The single chain antibody fragment comprises scFv. The single chain antibody fragment blocks assembly of the virus or bacteria by inhibiting expression of one or more viral or bacterial proteins.

The antibacterial molecule includes, inter alia, Peneidin-Like antimicrobial peptide AMP, among other antibacterial molecules.

The pathogens include virus, bacterium, protozoa, or poisons derived from algae, or a combination thereof. In one embodiment, the pathogens include Vibrio harveyi, White Spot Syndrome Virus, Taura, variants or serotypes thereof.

In another embodiment, the recombinant molecule is a DNA vaccine. The DNA vaccine can be codon optimized for expression in a specific microorganism and/or the target aquatic animal.

In yet another embodiment, the genetically modified microorganism is bioamplified in a probiotic organism prior to consumption by the aquatic animal.

The probiotic organism comprises bacterium, and planktonic organisms comprising Artemia, rotifers, copepods, or daphnia, or a combination thereof.

In one embodiment, the microorganism is a micro algae and transformation of micro algae is achieved by the genetic transformation of a symbiotic or commensal bacteria of the micro algae with a genetic material that expresses in vivo immunogenic peptides or antibody molecules against pathogenic infections of aquatic animal.

In one embodiment, the micro algae comprises Isochrysis, Pavlova, Nannochloropsis, Thalassiosira psuedonana, Cyanobacterium, Dunaliella, Phaeodactylum tricornutum, Red alga Porphydium cruentum, Haematococcus, Botryococcus, Gymnodinium sp; Gonyaulax, Chlamydomonas, Chlorella pyrenoidosa, or species and variants thereof. The cyanobacterium comprises Cyanobacterium Spirulina, cyanobacteria Scytonema, cyanobacteria Oscillatoria, or Synechococcus bacillarus, or species and variants thereof.

In one embodiment, the aquatic animal is a farm-raised or wild animal. In a preferred embodiment the aquatic animal is a farm-raised shrimp.

In another embodiment, the genetically modified micro algae expresses a protein, a peptide, or one or more antibody fragments that inhibit the growth or replication of a shrimp pathogen comprising Vibrio species, Taura, and White spot virus.

In yet another embodiment, the invention as described herein specifically excludes by way of proviso those methods for the prevention, amelioration or treatment of diseases or disorders in aquatic animals that use genetic transformation methods to generally boost the immune response of an aquatic animal to non-specific pathogens.

This invention as disclosed and described herein also expressly excludes the use of transformed insects or larvae thereof in delivering the genetic material or the recombinant molecules of the invention to the aquatic animal or to any intermediate hosts and/or feed organisms, including probiotic organisms, that are within the feeding cascade of the paratransgenesis methods of the invention.

These and other aspects and embodiments of the invention are disclosed in detail herein.

### IV. BRIEF DESCRIPTION OF THE FIGURES

FIG. 1: Schematic demonstrating the process of bioamplification. In this strategy, transgenic Dunaliella is initially consumed by feed organisms such as Artemia. The engorged Artemia is then fed to the target animal. In this manner, the supplement is bioamplified as it progresses up the food chain.

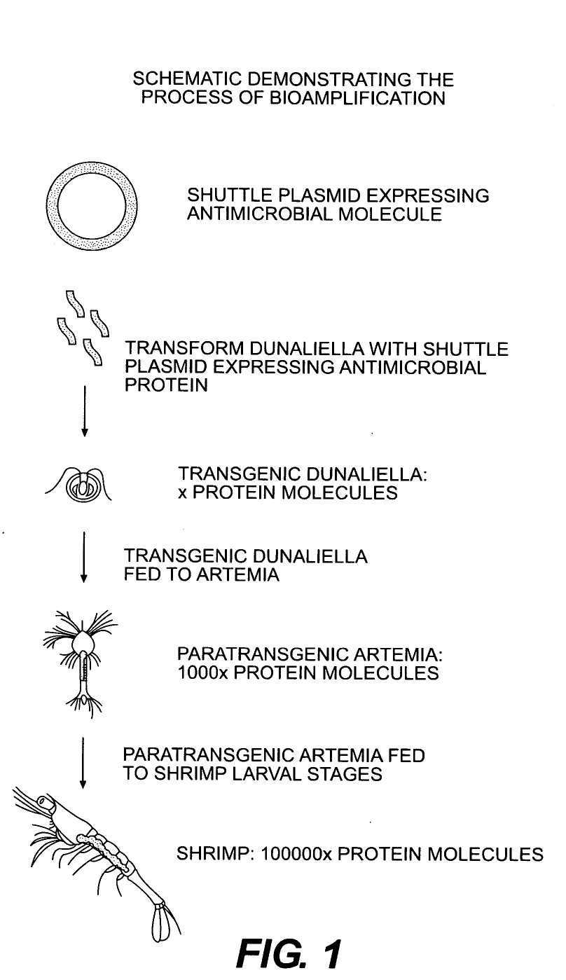

FIG. 2: A framework for a paratransgenic approach to control shrimp diseases. The cDNA encoding an anti-pathogen molecule is cloned into a shuttle vector (1,2), and expressed in E. coli. Plasmids carrying the recombinant DNA (3) is purified and subsequently used for transforming D. salina or another feed organism (4). The transgenic feed organisms is then fed to Artemia larvae (5). Artemia engorged with transgenic feed organisms (6) is then be used to feed shrimp larvae (7). Production of the anti-pathogen molecule within the gut of the shrimp protects the shrimp from targeted bacteria or viruses, resulting in healthy shrimp that is ready for harvest (8).

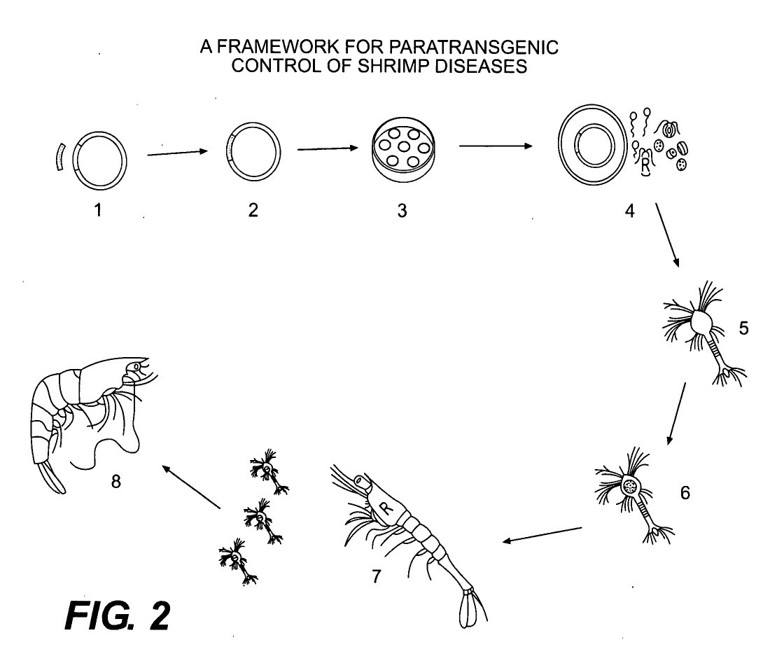

### V. DETAILED DESCRIPTION OF THE INVENTION

The invention as described and disclosed herein uses methods of paratransgenesis in order to control disease and disorders of target farmed and wild aquatic animals, to maintain equilibrium in the growth environment of these animals, and to efficiently transfer desirable genes and gene products to the target aquatic animals and their biological flora.

### DEFINITIONS

The definitions used in this application are for illustrative purposes and do not limit the scope of the invention.

As used herein, the term “micro algae” include both prokaryotic and eukaryotic algae that are classed in many different genera. Prokaryotic algae are typically referred to as cyanobacteria or blue-green algae. Eukaryotic micro algae come from many different genera, some of which overlap with the macro algae, but can be generally differentiated by their size and lack of defined organs. Micro algae can have specialized cell types. Examples of different groups containing micro algae include, but are not limited to, the Chlorophyta (e.g. Dunaliella), Phodophyta, Phaeophyta, Dinophyta, Euglenophyta, Cyanophyta, Prochlorophyta, and Cryptophyta.

The term microorganism has been used to include micro algae herein.

As used herein, the term “cyanobacteria” refers to prokaryotic organisms formerly classified as the blue-green algae. Cyanobacteria are a large and diverse group of photosynthetic bacteria which comprise the largest subgroup of Gram-negative bacteria. Cyanobacteria were classified as algae for many years due to their ability to perform oxygen-evolving photosynthesis. While many cyanobacteria have a mucilaginous sheath which exhibits a characteristic blue-green color, the sheaths in different species may also exhibit colors including light gold, yellow, brown, red, emerald green, blue, violet, and blue-black. Cyanobacteria include Microcystis aeruginosa, Trichodesmium erythraeum, Aphanizomenon flos-aquae, and Anabaena flos-aquae.

As used herein, the term “probiotic organisms” refers to organisms that act assist in amplification of the genetic material before being consumed by the target aquatic animal. Probiotic organisms include algae, bacteria, and fungi, such as yeast.

As used herein, the term “gene” or “genetic material” refers to an element or combination of elements that are capable of being expressed in a cell, either alone or in combination with other elements. In general, a gene comprises (from the 5′ to the 3′ end): (1) a promoter region, which includes a 5′ nontranslated leader sequence capable of functioning in prokaryotic and/or eukaryotic cells; (2) a structural gene or polynucleotide sequence, which codes for the desired protein; and (3) a 3′ nontranslated region, which typically causes the termination of transcription and the polyadenylation of the 3′ region of the RNA sequence. Each of these elements is operably linked by sequential attachment to the adjacent element. A gene comprising the above elements is inserted by standard recombinant DNA methods into a microorganism.

As used herein, “promoter” refers to a region of a DNA sequence active in the initiation and regulation of the expression of a structural gene. This sequence of DNA, usually upstream to the coding sequence of a structural gene, controls the expression of the coding region by providing the recognition for RNA polymerase and/or other elements required for transcription to start at the correct site.

As used herein, “protein” is used interchangeably with polypeptide, peptide and peptide fragments.

As used herein, “polynucleotide” includes cDNA, RNA, DNA/RNA hybrid, anti-sense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, included within the scope of the invention are alterations of a wild type or synthetic gene, including but not limited to deletion, insertion, substitution of one or more nucleotides, or fusion to other polynucleotide sequences, provided that such changes in the primary sequence of the gene do not alter the expressed peptide ability to elicit protective immunity.

As used herein, “recombinant molecule” includes any gene product that is produced in the course of the transcription, reverse-transcription, polymerization, translation, post-translation and/or expression of a gene. Recombinant molecules include, but are not limited to, proteins, polypeptides, peptides, peptide fragments, immunogenic peptides, fusion proteins, antibody fragments, polynucleotide molecules, DNA vaccine, among others.

As used herein, “vaccine” refers to compositions that result in both active and passive immunizations. Both pofynucleotides and their expressed gene products are referred as vaccines herein.

As used herein, “polypeptides” include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptide, homodimers, heterodimers, variants of the polypeptides, modified polypeptides, derivatives, analogs, fusion proteins, agonists, antagonists, or antibody of the polypeptide, among others. The polypeptides include natural peptides, recombinant peptides, synthetic peptides, or a combination thereof.

As used herein, the term “antibody fragments” refers to immunogenic or antigenic binding immunoglobulin peptides which are at least about 5 to about 15 amino acids or more in length, and which retain some biological activity or immunological activity of an immunoglobulin. The invention provides a novel approach to control of infectious diseases of commercial mariculture. In particular, the invention provides a method of delivering therapeutic molecules to an aquatic animal by methods of paratransgenesis that involves administration of a feed comprising transgenic micro algae, expressing a recombinant molecule that targets one or more key epitopes of a pathogen specific to the aquatic animal. Paratransgenesis employs genetically transformed microorganisms that are in symbiotic relationship with an intermediate host, or the target aquatic animal host. The microorganisms are closely linked to these hosts act as a ‘Trojan Horse’ to deliver neutralizing peptides and antibody fragments to the site of pathogen transmission within the host. An application of this method involves the expression of peptides and antibody fragments that specifically target key epitopes of pathogens of commercial mariculture and other types of aquaculture.

The transfer of the genetically modified microorganism to the target animal occurs through a natural biological process such as, for example, feeding the target animal with the transgenic microorganism directly or via bioamplification through probiotics.

Lines of marine cyanobacteria, algae and diatoms that are common components of feed for farmed shrimp and fish have been transformed to produce antibodies that neutralize infectious pathogens such as WSSV and Vibrio. Delivery of these feed organisms, either in slurry preparations or via a bioamplification strategy with a probiotic organism such as, for example, Artemia, resulted in passive immunization of the alimentary tract of farmed marine animals.

The microorganism used for a paratransgenic approach should satisfy the following requirements. The microorganism should be amenable to genetic manipulation, transformation of the microorganism should not alter their fitness, genetic manipulation of the microorganism should not affect its symbiotic functions in the host, or the ability of the host to consume the microorganism, the host that consumes or harbors the transgenic microorganism must maintain its growth and reproductive rates when compared to wild type controls, the products expressed by the transgenic microorganism should target the pathogens within the host, genetic modification of the microorganism should not render them virulent either to the host or other organisms in the environment, the microorganism chosen to be transformed should not be pathogenic to the host, strategies for foreign gene dispersal should target the host and selectively minimize non-target uptake and retention of the genetic material.

Probiotics are defined as micro-organisms that are beneficial to the health of the host. They are not therapeutic agents but, instead, directly or indirectly alter the composition of the microbial community in the rearing environment or in the gut of the host. Although the mode of action of probiotics is not fully understood, it is likely that they function by competitive elusion, that is, they antagonize the potential pathogen by the production of inhibitory compounds or by competition for nutrients and/or space. It is also likely that probiotics stimulate a humoral and/or cellular response in the host.

Probiotics are usually introduced as part of the feeding regimen or applied directly to the water. A variety of micro-organisms, ranging from aerobic Gram-positive bacteria (e.g., Bacillus spp), to Gram-negative bacteria (Vibro spp) and yeast have been utilized successfully to increase the commercial yield of farmed marine animals. Several species of micro algae have also effectively been used for this purpose. Of note, the unicellular algae, Tetraselmis suecica, has been used as feed for penaeids and salmonids with significant reduction in the level of bacterial diseases. The antagonism among microorganisms is a naturally occurring phenomenon through which pathogens can be killed or reduced in number in the aquaculture environment. In aquaculture, where micro algae are used as the main live food, the survival rates of prawns, crabs and finfish are not considered to be sufficiently high. However, if certain species of bacteria are present with the algae, the survival rate increases significantly. It is therefore preferable to feed microorganisms to fish along with algae, although the control of these microorganisms is essential to prevent the pathogens from dominating the microbial communities. These results have led to further studies using viruses, fungi and protozoa as biocontrol agents to eliminate pathogenic organisms. The paratransgenesis method of the invention complements biocontrol strategies at a molecular level in preventing or treating infectious diseas of aquaculture while maintaining the natural balance in their habitat, helping to maintain suitable environmental conditions in aquaculture and promoting the growth and health of aquaculture in a most efficient and environmentally friendly manner.

Paratransgenic methods of the invention demonstrate environmentally acceptable approaches for control of marine effective control of infections in mariculture and offer robust and pathogen transmission. The risk assessment framework being developed for paratransgenic control of arthropod-borne diseases can be applied in part to mariculture. Unique aspects of the marine environment, such as novel microflora and fauna, physical and chemical features of marine ecosystems and complex interactions through marine food chains were modeled and evaluated carefully during development of paratransgenic interventions.

Pathogens within the scope of the invention include a wider variety of agents that specifically infect mariculture. Pathogens include viral or bacterial pathogens as well as toxins produced by algae such as, for example, dinoflagellates. These pathogens include, by way of example and not limitations, White Spot Syndrome Virus (WSSV), species of Vibrio (including V. anguillarum and V. ordalii, Vibrio salmonicida, Vibrio harveyi), causative agents and virus for infectious hypodermal and haematopoietic necrosis (IHHN) and IHHNV, causative agent for run-deformity syndrome or RDS of Penaeus vannamei, Baculo-like viruses, Infectious Pancreatic Necrosis Virus (IPNV), Hirame rhabdovirus (HIRRV), the Yellowtail Ascites Virus (YAV), Striped Jack Nervous Necrosis Virus (SJNNV), Irido, Aeromonos hydrophila, Aeromonos salmonicida, Serratia liquefaciens, Yersnia ruckeri type I, Infectious salmon anaemia (USA) virus, Pancreas Disease (PD), Viral Hemorrhagic Septicemia (VHS), Rennibacterium salmoninarum, Aeromonas salmonicida, Aeromonas hydrophila, species of Pasteurella (including P. piscicida), species of Yersinia, species of Streptococcus, Edwardsiella tarda and Edwardsiella ictaluria; the viruses causing viral hemorrhagic septicemia, infectious pancreatic necrosis, viremia of carp, channel catfish virus, grass carp hemorrhagic virus, nodaviridae such as nervous necrosis virus, infectious salmon anaemia virus; and the parasites Ceratomyxa shasta, Ichthyophthirius multifillius, Cryptobia salmositica, Lepeophtherius salmonis, Tetrahymena species, Trichodina species and Epistylus species, dinoflagellates toxins including toxins causing Diaarhetic Shellfish Poisoning (DSP), Paralytic Shellfish Poisoning (PSP), Neurotoxin poisoning (NSP) and Ciguatera, and many more, all of which cause serious damage in aquaculture.

In a preferred embodiment, the method of the invention employs genetically transformed cyanobacteria or Dunaliella that express P. monodon antiviral protein (PmAV) and P. monodon antimicrobial peptide (PmAMP).

Aquatic animals includes vertebrates, invertebrates, arthropods, fish, mollusks, including, by way of example and not limitation, shrimp (e.g., penaeid shrimp, brine shrimp, freshwater shrimp, etc), crabs, oysters, scallop, prawn clams, cartilaginous fish (e.g., bass, striped bass, tilapia, catfish, sea bream, rainbow trout, zebrafish, red drum, salmonids, carp, catfish, yellowtail, carp, etc), crustaceans, among others. Shrimp includes all variety and species of shrimp, including by way of example and not limitation, Penaeus stylirostris, Penaeus vannamei, Penaeus monodon, Penaeus chinensis, Penaeus occidentalis, Penaeus californiensis, Penaeus semisulcatus, Penaeus monodon, Penaeus esculentu, Penaeus setiferus, Penaeus japonicus, Penaeus aztecus, Penaeus duorarum, Penaeus indicus, and Penaeus merguiensis, among others species of shrimp.

Expression Vectors

Also encompassed within the scope of the invention are expression vectors containing the gene constructs of the invention. Expression vectors are defined herein as DNA sequences that are required for the transcription of cloned copies of genes and the translation of their mRNAs in an appropriate host. Such expression vectors are used to express eukaryotic and prokaryotic genes in a variety of hosts such as bacteria, yeast, plant cells, fungi, insect cells and animal cells. Expression vectors include, but are not limited to, cloning vectors, modified cloning vectors, specifically, designed plasmids or viruses.

According to one embodiment of the invention described herein, there are provided expression vectors containing one or more gene constructs of the invention carrying the antibody genes, including antibody subunit genes or fragments thereof. The expression vectors of the invention contain the necessary elements to accomplish genetic transformation of microorganisms so that the gene constructs are introduced into the microorganism's genetic material in a stable manner, i.e., a manner that will allow the antibody genes to be passed on the microorganism's progeny. The design and construction of the expression vectors influence the integration of the gene constructs into the microorganism genome and the ability of the antibody genes to be expressed by microorganism cells.

Preferred among expression vectors are vectors carrying a functionally complete human or mammalian heavy or light chain sequence having appropriate restriction sites engineered so that any variable V_{H} or variable V_{L} chain sequence with appropriate cohesive ends can be easily inserted therein. Human C_{H} or C_{L} chain sequence-containing vectors are thus an embodiment of the invention and can be used as intermediates for the expression of any desired complete H or L chain in any appropriate host.

Many vector systems are available for the expression of cloned HC and LC genes in host cells. Different approaches can be followed to obtain complete HC and LC subunit antibodies. In one embodiment, HC and LC were co-expressed in the same cells to achieve intracellular association and linkage of HC and LC into complete tetrameric HC and LC antibodies. The co-expression can occur by using either the same or different plasmids in the same host.

Polynucleotides encoding both HC and LC are placed under the control of one or more different or the same promoters, for example in the form of a dicistronic operon, into the same or different expression vectors. The expression vectors are then transformed into cells, thereby selecting directly for cells that express both chains.

In one embodiment, the polynucleotide encoding LC and polynucleotides encoding HC are present on two mutually compatible expression vectors which are each under the control of different or the same promoter(s). In this embodiment, the expression vectors are co-transformed or transformed individually. For example, cells are transformed first with an expression vector encoding one chain, for example LC, followed by transformation of the resulting cell with an expression vector encoding a iiC.

In another embodiment, a single expression vector carrying polynucleotides encoding both the HC and LC is used. Cell lines expressing HC and LC molecules could be transformed with expression vectors encoding additional copies of LC, HC, or LC plus HC in conjunction with additional selectable markers to generate cell lines with enhanced properties, such as higher production of assembled HC and LC antibody molecules or enhanced stability of the transformed cell lines.

Specifically designed expression vectors allow the shuttling of DNA between hosts, such as between bacteria-plant or bacteria-animal cells. According to a preferred embodiment of the invention, the expression vector contains an origin of replication for autonomous replication in host cells, selectable markers, a limited number of useful restriction enzyme sites, active promoter(s), and additional regulatory control sequences.

Preferred among expression vectors, in certain embodiments, are those expression vectors that contain cis-acting control regions effective for expression in a host operatively linked to the polynucleotide of the invention to be expressed. Appropriate trans-acting factors are supplied by the host, supplied by a complementing vector or supplied by the vector itself upon introduction into the host.

In certain preferred embodiments in this regard, the expression vectors provide for specific expression. Such specific expression is an inducible expression, cell or organ specific expression, host-specific expression, or a combination thereof.

Promoters

Promoters are responsible for the regulation of the transcription of DNA into mRNA. A number of promoters which function in microorganism cells are known in the art, and may be employed in the practice of the present invention. These promoters are obtained from a variety of sources such as, for example, viruses, plant, and bacteria, among others.

The invention, as described and disclosed herein, encompasses the use of constitutive promoters, inducible promoters, or both. In general, an “inducible promoter” is a promoter that is capable of directly or indirectly activating transcription of one or more DNA sequences or genes in response to an inducer. In the absence of an inducer the DNA sequences or genes will not be transcribed. Typically the protein factor that binds specifically to an inducible promoter to activate transcription is present in an inactive form which is then directly or indirectly converted to the active form by the inducer. The inducer can be a chemical agent such as a protein, metabolite, growth regulator, herbicide or phenolic compound or a physiological stress imposed directly by heat, cold, wound, salt, or toxic elements, light, desiccation, pathogen infection, or pest-infestation.

Inducible promoters are determined using any methods known in the art. For example, the promoter may be operably associated with an assayable marker gene such as GUS (glucouronidase), the host microorganism can be engineered with the construct; and the ability and activity of the promoter to drive the expression of the marker gene in the harvested tissue under various conditions assayed.

A microorganism cell containing an inducible promoter is exposed to an inducer by externally applying the inducer to the cell or microorganism such as by spraying, harvesting, watering, heating or similar methods. A “constitutive promoter” is a promoter that directs the expression of a gene throughout the various parts of an organism and continuously throughout development of the organism.

In one embodiment of the invention, promoters are tissue-specific. Non-tissue-specific promoters (i.e., those that express in all tissues after induction), however, are preferred. More preferred are promoters that additionally have no or very low activity in the uninduced state. Most preferred are promoters that additionally have very high activity after induction. Particularly preferred among inducible promoters are those that can be induced to express a protein by environmental factors that are easy to manipulate.

In a preferred embodiment of the invention, one or more constitutive promoters are used to regulate expression of the antibody genes or antibody subunit genes in microorganisms.

Examples of an inducible and/or constitutive promoters include, but are not limited to, promoters isolated from the caulimovirus group such as the cauliflower mosaic virus 35S promoter (CaMV35S), the enhanced cauliflower mosaic virus 35S promoter (enh CaMV35S), the figwort mosaic virus full-length transcript promoter (FMV35S), the promoter isolated from the chlorophyll a/b binding protein, proteinase inhibitors (PI-I, PI-II), defense response genes, phytoalexin biosynthesis, phenylpropanoid phytoalexin, phenylalanine ammonia lyase (PAL), 4-coumarate CoA ligase (4CL), chalcone synthase (CHS), chalcone isomerase (CHI), resveratrol (stilbene) synthase, isoflavone reductase (IFR), terpenoid phytoalexins, HMG-CoA reductase (HMG), casbene synthetase, cell wall components, lignin, phenylalanine ammonia lyase, cinnamyl alcohol dehydrogenase (CAD), caffeic acid o-methyltransferase, lignin-forming peroxidase, hydroxyproline-rich glycoproteins (HRGP), glycine-rich proteins (GRP), thionins, hydrolases, lytic enzymes, chitinases (PR-P, PR-Q), class I chitinase, basic, Class I and II chitinase, acidic, class II chitinase, bifunctional lysozyme, β-1,3-Glucanase, arabidopsis, β-fructosidase, superoxide dismutase (SOD), lipoxygenase, prot., PR1 family, PR2, PR3, osmotin, PR5, ubiquitin, wound-inducible genes, win1, win2 (hevein-like), wun1, wun2, nos, nopaline synthase, ACC synthase, HMG-CoA reductase hmg1, 3-deoxy-D-arabino-heptulosonate-7-phosphate synthase, HSP7033, Salicylic acid inducible, acid peroxidase, PR-proteins, glycine-rich protein, methyl jasmonate inducible, vspB⁴², heat-shock genes, HSP70, cold-stress inducible, drought, salt stress, hormone inducible, gibberellin, α-amylase, abscisic acid, EM-1, RAB, LEA genes, ethylene, phytoalexin biosyn genes, or a combination thereof among others.

The above-noted promoters are listed solely by way of illustration of the many commercially available and well known promoters that are available to those of skill in the art for use in accordance with this aspect of the present invention. It will be appreciated that any other promoter suitable for, for example, introduction, maintenance, propagation or expression of a polynucleotide or polypeptide of the invention in microorganism may be used in this aspect of the invention.

Regulatory Control Elements

Gene constructs or genetic material of the present invention can also include other optional regulatory elements that regulate, as well as engender, expression. Generally such regulatory control elements-operate by controlling transcription. Examples of such regulatory control elements include, for example, enhancers (either translational or transcriptional enhancers as may be required), repressor binding sites, terminators, leader sequences, and the like.

Specific examples of these elements include, the enhancer region of the 35S regulatory region, as well as other enhancers obtained from other regulatory regions, and/or the ATG initiation codon and adjacent sequences. The initiation codon must be in phase with the reading frame of the coding sequence to ensure translation of the entire sequence. The translation control signals and initiation codons are from a variety of origins, both natural and synthetic. Translational initiation regions are provided from the source of the transcriptional initiation region, or from the structural gene. The sequence is also derived from the promoter selected to express the gene, and can be specifically modified to increase translation of the mRNA.

The nontranslated leader sequence is derived from any suitable source and is specifically modified to increase the translation of the mRNA. In one embodiment, the 5′ nontranslated region is obtained from the promoter selected to express the gene, the native leader sequence of the gene, coding region to be expressed, viral RNAs, suitable eucaryotic genes, or a synthetic gene sequence, among others.

In another embodiment, gene constructs of the present invention comprise a 3U untranslated region. A 3U untranslated region refers to that portion of a gene comprising a DNA segment that contains a polyadenylation signal and any other regulatory signals capable of effecting mRNA processing or gene expression. The polyadenylation signal is usually characterized by effecting the addition of polyadenylic acid tracks to the 3U end of the mRNA precursor.

The termination region or 3′ nontranslated region is employed to cause the termination of transcription and the addition of polyadenylated ribonucleotides to the 3′ end of the transcribed mRNA sequence. The termination region may be native with the promoter region, native with the structural gene, or may be derived from the expression vector or another source, and would preferably include a terminator and a sequence coding for polyadenylation. The addition of appropriate introns and/or modifications of coding sequences for increased translation can also substantially improve foreign gene expression.

Selectable Markers

To aid in identification of transformed microorganism cells, the gene constructs of this invention may be further manipulated to include selectable marker genes that are functional in bacteria, algae, and/or aquatic host. Useful selectable markers include, but are not limited to, enzymes which provide for resistance to an antibiotic such as Ampicillin resistance gene (Amp^{r}), tetracycline resistance gene (Tcr), Cycloheximide-resistance L41 gene, the gene conferring resistance to Antibiotic G418 such as the APT gene derived from a bacterial transposon Tn903, the antibiotic Hygromycin B-resistance gene, Gentamycin resistance gene, and/or kanamycine resistance gene, among others. Similarly, enzymes providing for production of a compound identifiable by color change such as GUS, or luminescence, such as luciferase are included herein.

A selectable marker gene is used to select transgenic microorganism cells of the invention, which transgenic cells have integrated therein one or more copies of the gene construct of the invention. The selectable or screenable genes provide another control for the successful culturing of cells carrying the genes of interest. Transformed microorganism may be selected by growing the cells on a medium containing, for example, Kanamycin.

Transformation Strategies

Microorganisms are genetically transformed to incorporate one or more gene constructs of the invention. There are numerous factors which influence the success of transformation. The design and construction of the expression vector influence the integration of the foreign genes into the genome of the microorganism and the ability of the foreign genes to be expressed by the microorganism. The integration of the polynucleotides encoding the desired gene into the microorganism is achieved through strategies that involve, for example, insertion or replacement methods. These methods involve strategies utilizing, for example, direct terminal repeats, inverted terminal repeats, double expression cassette knock-in, specific gene knock-in, specific gene knock-out, random chemical mutagenesis, random mutagenesis via transposon, and the like. The expression vector is, for example, flanked with homologous sequences of any non-essential microorganism genes, transposon sequence, or ribosomal genes. The DNA is then integrated in host by homologous recombination occurred in the flanking sequences using standard techniques.

In other embodiments, various alternative methods for introducing recombinant nucleic acid constructs into microorganisms are also utilized. Alternative gene transfer and transformation methods include, but are not limited to, electroporation-mediated uptake of naked DNA, microinjection, silicon carbide mediated DNA uptake, and microprojectile bombardment, among others.

In the case of direct gene transfer, the gene construct is transformed into microorganism without the use of plasmids. Direct transformation involves the uptake of exogenous genetic material into microorganism. Such uptake may be enhanced by use of chemical agents or electric fields. The exogenous material may then be integrated into the nuclear genome. Alternatively, exogenous DNA can be introduced into cells or by microinjection. In this technique, a solution of the plasmid DNA or DNA fragment is injected directly into the cell with a finely pulled glass needle. A more recently developed procedure for direct gene transfer involves bombardment of cells by micro-projectiles carrying DNA. In this procedure, commonly called particle bombardment, tungsten or gold particles coated with the exogenous DNA are accelerated toward the target cells. The particles penetrate the cells carrying with them the coated DNA. Microparticle acceleration has been successfully demonstrated to lead to both transient expression and stable expression in cells suspended in cultures.

Use of Vaccines in Mariculture

The use of antibodies for therapeutic and diagnostic purposes has gained prominence in the past decade. Immunoglobulins are very specific to their targets and could be used to design high affinity-based reagents for immunotherapeutic applications. Problems associated with the relatively short half life of passively administered immunoglobulins can be overcome by using constitutively-expressed single chain antibodies (scFv), instead of whole IgG molecules. These are smaller in size and can be synthesized as bivalent to multivalent molecules that can attack different targets on the pathogen.

The invention provides for genetic materials that encode antibody fragments that are expressed within the microorganism before consumption by the aquatic animal. In one embodiment, the antibodies include immunoglobulin molecules having H and L chains associated therein so that the overall molecule exhibits the desired binding and recognition properties. Various types of immunoglobulin molecules are provided: monovalent, divalent, or molecules with the specificity-determining V binding domains attached to moieties carrying desired functions.

In another embodiment, the invention provides for genetic material encoding fragments of chimeric immunoglobulin molecules such as Fab, Fab′, or F(ab′)₂ molecules or those proteins coded by truncated genes to yield molecular species functionally resembling these fragments. A chimeric chain contains a constant (C) region substantially similar to that present in a natural mammalian immunoglobulin, and a variable (V) region having the desired anti-pathogenic specificity of the invention. Antibodies having chimeric H chains and L chains of the same or different V region binding specificity are prepared by appropriate association of the desired polypeptide chains.

The immunoglobulin molecules are encoded by genes which include the kappa, lambda, alpha, gamma, delta, epsilon and mu constant regions, as well as any number of immunoglobulin variable regions. Light chains are classified as either kappa or lambda. Light chains comprise a variable light (V_{L}) and a constant light (C_{L}) domain. Heavy chains are classified as gamma, mu, alpha, delta, or epsilon, which in turn define the immunoglobulin classes IgG, IgM, IgA, IgD and IgE, respectively. Heavy chains comprise variable heavy (V_{H}), constant heavy 1 (CH₁), hinge, constant heavy 2 (CH₂), and constant heavy 3 (CH₃) domains. The mammalian IgG heavy chains are further sub-classified based on their sequence variation, and the subclasses are designated IgG1, IgG2, IgG3 and IgG4.

Antibodies can be further broken down into two pairs of a light and heavy domain. The paired V_{L} and V_{H} domains each comprise a series of seven subdomains: framework region 1 (FR1), complementarity determining region 1 (CDR1), framework region 2 (FR2), complementarity determining region 2 (CDR2), framework region 3 (FR3), complementarity determining region 3 (CDR3), framework region 4 (FR4) which constitute the antibody-antigen recognition domain, etc.

In general, as used herein, the term antibody or antibody fragment of the invention encompasses variety of modifications, particularly those that are present in polypeptides expressed by polynucleotides in a host cell. It will be appreciated that polypeptides often contain amino acids other than the 20 amino acids commonly referred to as the 20 naturally occurring amino acids, and that many amino acids, including the terminal amino acids, may be modified in a given polypeptide, either by natural processes, such as processing and other post-translational modifications, or by chemical modification techniques.

Modifications occur anywhere in a polypeptide, including the peptide backbone, the amino acid side chains and the amino or carboxyl termini. Blockage of the amino or carboxyl group in a polypeptide, or both, by a covalent modification, occurs in natural or synthetic polypeptides and such modifications may be present in the antibody polypeptides of the present invention, as well. In general, the nature and extent of the modifications are determined by the host cell's post-translational modification capacity and the modification signals present in the polypeptide amino acid sequence. It will be appreciated that the same type of modification may be present in the same or varying degrees at several sites in a polypeptide.

The microorganism-derived antibody according to the invention includes truncated and/or N-terminally or C-terminally extended forms of the antibody, analogs having amino acid substitutions, additions and/or deletions, allelic variants and derivatives of the antibody. Variations in the structure of microorganism-derived antibodies may arise naturally as allelic variations, as disclosed above, due to genetic polymorphism, for example, or may be produced by human intervention (i.e., by mutagenesis of cloned DNA sequences), such as induced point, deletion, insertion and substitution mutants. Minor changes in amino acid sequence are generally preferred, such as conservative amino acid replacements, small internal deletions or insertions, and additions or deletions at the ends of the molecules.

It has been demonstrated that human monoclonal antibodies can be expressed in transgenic algae chloroplasts. C. reinhardtii chloroplast atpA or rbcL promoters and its 5′ untranslated regions were used to drive expression of an engineered large single-chain antibody gene in this algae. This antibody is directed against herpes simplex virus (HSV) glycoprotein D and accumulates as a functional soluble protein in transgenic chloroplasts, and binds herpes virus proteins, as determined by ELISA assays. These studies demonstrated that algae can be used as an expression platform to synthesize complex recombinant proteins.

Costs for production of recombinant proteins in algal systems are quite reasonable ($0.002 per liter). In addition, algae can be grown in continuous culture, their growth medium can be recycled, transgenic algae can be generated quickly, as it requires only a few weeks between the generation of initial transformants and their scale-up to production volumes, and finally, the chloroplast and nuclear genome of algae can be genetically transformed opening the possibility of producing any transgenic protein in a single organism.

Chimeric antibody technology bridges both the hybridoma and genetic engineering technologies to provide recombinant molecules for the prevention and treatment of infections in marine culture. The chimeric antibodies of the present invention embody a combination of the advantageous characteristics of mAbs. Like mouse mAbs, they can recognize and bind to a specific epitope of an antigen present in the target animal. Moreover, using the methods disclosed in the present invention, any desired antibody isotype can be combined with any particular antigen combining site.

In one embodiment, the invention provides cyanobacteria or micro algae-derived mammalian or chimeric antibodies, including antibody subunits and fragments thereof, with specificity to a pathogen of marinculture.

In another embodiment, Synechococcus bacillarus, a cyanobacterium, was transformed with a DNA construct that encodes a single chain antibody and can stably express the corresponding scFv in its functional state. In this study, an expression plasmid encoding the murine single chain antibody, rDB3 was used (Durvasula et al. 1999, incorporated herein by reference in its entirety). This study confirms that genetically modified cyanobacteria can be used as a delivery system to secrete anti-pathogen molecules that affect shrimp and mollusks, as part of a paratransgenic strategy to control infectious diseases of mariculture.

In another embodiment, immune peptides and antibody fragments were expressed in transgenic Chlorophyta spp. In particular, single chain antibody fragments were developed that target key epitopes of marine pathogens such as, for example, Vibrio spp, White Spot Syndrome Virus (WSSV), or a combination thereof, among others. Central to all of our paratransgenic approaches is the concept of co-expression of multiple immune peptides and antibody fragments, to minimize evolution of resistance amongst target pathogens. As delivery systems are refined to disperse engineered algae and cyanobacteria into populations of shrimp, shellfish and fish, the invention has deployed multiple strains of engineered organisms that target unique pathogen epitopes.

Expression of immune peptides and engineered single chain antibody fragments by organisms that are used as feed in mariculture operations offers a novel strategy to deliver passive immunity to the gut of farmed shrimp, shellfish and fish. Since many pathogens gain access via the digestive tract, this approach enhances the arsenal against several infections that currently afflict mariculture operations.

Polynucleotides Encoding Antibody Polypeptides This invention also encompasses polynucleotides that correspond to and code for the antibody polypeptides. Nucleic acid sequences are either synthesized using automated systems well known in the art, or derived from a gene bank.

It will be appreciated that a great variety of modifications have been made to DNA and RNA that serve many useful purposes known to those of skill in the art. The polynucleotides of the invention embrace chemically, enzymatically or metabolically modified forms of polynucleotides.

The polynucleotides of the present invention encode, for example, the coding sequence for the structural gene (i.e., antibody gene), and additional coding or non-coding sequences. Examples of additional coding sequences include, but are not limited to, sequences encoding a secretory sequence, such as a pre-, pro-, or prepro-protein sequences. Examples of additional non-coding sequences include, but are not limited to, introns and non-coding 5′ and 3′ sequences, such as the transcribed, non-translated sequences that play a role in transcription and mRNA processing, including splicing and polyadenylation signals, for example, for ribosome binding and stability of mRNA.

The polynucleotides of the invention also encode a polypeptide which is the mature protein plus additional amino or carboxyl-terminal amino acids, or amino acids interior to the mature polypeptide (when the mature form has more than one polypeptide chain, for instance). Such sequences play a role in, for example, processing of a protein from precursor to a mature form, may facilitating protein trafficking, prolonging or shortening protein half-life or facilitating manipulation of a protein for assay or production, among others. The additional amino acids may be processed away from the mature protein by cellular enzymes.

In sum, the polynucleotides of the present invention encode, for example, a mature protein, a mature protein plus a leader sequence (which may be referred to as a preprotein), a precursor of a mature protein having one or more prosequences which are not the leader sequences of a preprotein, or a preproprotein, which is a precursor to a proprotein, having a leader sequence and one or more prosequences, which generally are removed during processing steps that produce active and mature forms of the polypeptide.

The polynucleotides of the invention include “variant(s)” of polynucleotides, or polypeptides as the term is used herein. Variants include polynucleotides that differ in nucleotide sequence from another reference polynucleotide. Generally, differences are limited so that the nucleotide sequences of the reference and the variant are closely similar overall and, in many regions, identical. As noted below, changes in the nucleotide sequence of the variant may be silent. That is, they may not alter the amino acids encoded by the polynucleotide. Where alterations are limited to silent changes of this type, a variant will encode a polypeptide with the same amino acid sequence as the reference.

Changes in the nucleotide sequence of the variant may alter the amino acid sequence of a polypeptide encoded by the reference polynucleotide. Such nucleotide changes may result in amino acid substitutions, additions, deletions, fusions and truncations in the polypeptide encoded by the reference sequence. According to a preferred embodiment of the invention, there are no alterations in the amino acid sequence of the polypeptide encoded by the polynucleotides of the invention, as compared with the amino acid sequence of the wild type or mammalian derived peptide.

The present invention further relates to polynucleotides that hybridize to the herein described sequences. The term “hybridization under stringent conditions” according to the present invention is used as described by Sambrook et al. (1989) Molecular Cloning, A Laboratory Manual, Cold Spring Harbor Laboratory Press 1.101-1.104. Preferably, a stringent hybridization according to the present invention is given when after washing for an hour with 1% SSC and 0.1% SDC at 50° C., preferably at 55° C., more preferably at 62° C., most preferably at 68° C., a positive hybridization signal is still observed. A polynucleotide sequence which hybridizes under such washing conditions with the nucleotide sequence shown in any sequence disclosed herein or with a nucleotide sequence corresponding thereto within the degeneration of the genetic code is a nucleotide sequence according to the invention.

The polynucleotides of the invention include polynucleotide sequences that have at least about 50%, 65%, 70%, 75%, 80%, 85%, 90%, 95%, 98%, 99% or more nucleotide sequence identity to the polynucleotides or a transcriptionally active fragment thereof. To determine the percent identity of two amino acid sequences or two nucleic acid sequences, the sequences are aligned for optimal comparison purposes (i.e., gaps can be introduced in the sequence of a first amino acid or nucleic acid sequence for optimal alignment with a second nucleic acid sequence). The amino acid residue or nucleotides at corresponding amino acid or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical overlapping positions/total # of positions×100). In one embodiment, the two sequences are the same length.

The determination of percent identity between two sequences also can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST program of Altschul et al. (1990), J. Mol. Biol. 215:403-410. BLAST nucleotide searches can be performed with the NBLAST program, score=100, wordlength 12 to obtain nucleotide sequences homologous to a nucleic acid molecules of the invention. The BLAST protein searches can be performed with the XBLAST program, score=50, wordlength=3 to obtain amino acid sequences homologous to a protein molecule of the invention. To obtain gapped alignments for comparison purposes, Gapped BLAST can be utilized as described in Altschul et al (1997) Nucleic Acids Res. 25:3389-3402.

Alternatively, PSI-Blast can be used to perform an iterated search which detects distant relationships between molecules (Id.). When utilizing BLAST, Gapped BLAST and PSI-Blast programs, the default parameters of the respective programs (i.e., XBLAST and NBLAST program can be used. Another preferred, non-limiting example of a mathematical algorithm utilized for the comparison of sequences is the algorithm of Myers and Miller (1988)CABIOS 4:11-17. Such an algorithm is incorporated into the ALIGN program (version 2.0) which is part of the GCG sequence alignment software package. When utilizing the ALIGN program for comparing amino acid sequences of a PAM 120 weight residue table, a gap length penalty of 12 and a gap penalty of 4 can be used. In an alternate embodiment, alignments can be obtained using the NA_MULTIPLE_ALIGNMENT 1.0 program, using a GapWeight of 5 and a GapLength Weight of 1.

DNA Vaccines

According to one embodiment of the invention, the recombinant molecule is a DNA vaccine. DNA vaccines, an alternative to a traditional vaccine comprising an antigen and an adjuvant, involve the direct in vivo introduction of DNA encoding the antigen into tissues of an organism for expression of the antigen by the cells of the subject's organism. Such vaccines are termed herein “DNA vaccines” or “polynucleotide-based vaccines” DNA vaccines are described in International Patent Publication WO 95/20660 and International Patent Publication WO 93/19183, the disclosures of which are hereby incorporated by reference in their entireties.

In contrast to conventional vaccines, DNA and other subunit vaccines exclusively utilize host cell molecules for transcription and translation of proteins. In one embodiment, the DNA vaccine of the invention contains modified codon usage of the host micro algae.

The ability of directly injected DNA that encodes a protein to elicit a protective immune response has been demonstrated in numerous experimental systems Conry et al., Cancer Res., 54:1164-1168 (1994); Cox et al., Virol, 67:5664-5667 (1993); Davis et al., Hum. Mole. Genet., 2:1847-1851 (1993); Sedegah et al., Proc. Natl. Acad. Sci., 91:9866-9870 (1994); Montgomery et al., DNA Cell Bio., 12:777-783 (1993); Ulmer et al., Science, 259:1745-1749 (1993); Wang et al., Proc. Natl. Acad. Sci., 90:4156-4160 (1993); Xiang et al., Virology, 199:132-140 (1994).

Studies with ferrets indicate that DNA vaccines against conserved internal viral proteins of influenza, together with surface glycoproteins, are more effective against antigenic variants of influenza virus than are either inactivated or subvirion vaccines [Donnelly et al., Nat. Medicine, 6:583-587 (1995)].

One of the advantages of DNA immunization over antigen immunization is the potential for the immunogen to enter the MHC class I pathway and evoke a cytotoxic T cell response. Immunization of mice with DNA encoding the influenza A nucleoprotein (NP) elicited a CD8+ response to NP that protected mice against challenge with heterologous strains of flu. (Montgomery, et al., supra). Another advantage of the immunization with a DNA vaccine rather than its gene product is the relative simplicity with which native or nearly native antigen can be presented to the immune system. Mammalian proteins expressed recombinantly in bacteria, yeast, or even mammalian cells often require extensive treatment to ensure appropriate immunogenicity.

Also, the ease of producing and purifying DNA constructs compares favorably with traditional protein purification, facilitating the generation of combination vaccines. Thus, in accordance with one embodiment of the invention, multiple genes, for example, genes encoding antigens VP19+VP28 of WSSV in combination with other genes encoding any other viral or bacterial binding antigens are transferred to the micro algae according to the methods of the invention.

As is well known in the art, a large number of factors can influence the efficiency of expression of antigen genes and/or the immunogenicity of DNA vaccines. Examples of such factors include the reproducibility of inoculation, construction of the plasmid vector, choice of the promoter used to drive antigen gene expression and stability of the inserted gene in the plasmid. Depending on their origin, promoters differ in tissue specificity and efficiency in initiating mRNA synthesis (see, for example, Xiang et al., Virology, 209:564-579 (1994); Chapman et. al., Nucleic Acids. Res., 19:3979-3986 (1991). To date, most DNA vaccines in mammalian systems have relied upon viral promoters derived from cytomegalovirus (CMV).

Another factor known to affect the immune response elicited by DNA immunization is the method of DNA delivery. For example, high-velocity inoculation of plasmids, using a gene-gun, enhanced the immune responses of mice, Eisenbraun et al., DNA Cell Biol., 12: 791-797 (1993), presumably because of a greater efficiency of DNA transfection and more effective antigen presentation by dendritic cells.

Also contemplated within the scope of the invention is the use of naked polynucleotides, unassociated with any plasmids, proteins, adjuvants or other agents which affect the recipients' immune system. In this case, it is desirable for the polynucleotide to be in a physiologically acceptable solution, such as, but not limited to, sterile saline or sterile buffered saline. Alternatively, the polynucleotides may be associated with liposomes, such as lecithin liposomes or other liposomes known in the art, as a DNA-liposome mixture, or associated with an adjuvant known in the art to boost immune responses, such as a protein or other carrier.

Agents that assist in the cellular uptake of DNA, such as, but not limited to, calcium ions, may also be used. These agents are generally referred to herein as transfection facilitating reagents and pharmaceutically acceptable carriers. Techniques for coating microprojectiles coated with polynucleotides are known in the art and are also useful in connection with this invention.

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLES

### Example 1

### Transformation of Dunliella

This example demonstrates the use of Dunaliella for paratransgenic control of infectious diseases of farmed shrimp. Paratransgenesis is a strategy that employs commensal or symbiotic organisms to express molecules that interfere with transmission cycles of infectious pathogens. Application of paratransgenesis to viral and bacterial diseases of farmed shrimp has been described with genetically modified cyanobacteria as the delivery agents. Here, a transformation system for Dunaliella and a framework for application of engineered Dunaliella in commercial mariculture are presented.

D. salina was transformed carrying a construct that contains the chloramphenicol acetyltransferase (CAT) gene as well as the gene that encodes for green fluorescent protein (GFP). Using GFP as a marker, we fed the transformed Dunaliella as a slurry to shrimp nauplii and monitor the progression of the GFP within the gut. This study demonstrated successful delivery of a functional protein from the feed to the target organism.

Dunaliella salina is also being transformed to express molecules with activity against infectious pathogens of mariculture. A gene encoding Penaeidin, an antibacterial peptide with activity against Vibrio species, has been synthesized and is incorporated into an expression plasmid for transformation of Dunaliella. Extracts from transformed lines of D. salina have been analyzed for activity against a variety of gram negative and gram positive bacteria, with special focus on Virio harveyii.

### Example 2

### Transgenic Microorganism Expressing Antibody Fragments

Lines of marine cyanobacteria, algae and diatoms—common components of feed for farmed shrimp and fish—were transformed to produce antibodies that neutralize infectious pathogens such as WSSV and Vibrio. Delivery of these feed organisms, either in slurry preparations or via a bioamplification strategy with Artemia, results in passive immunization of the alimentary tract of farmed marine animals.

This is the portal of entry for many infectious agents and the delivery of neutralizing antibodies would either abort the infectious process or delay it sufficiently to permit harvest. We have demonstrated that a marine cyanobacterium, Synechococcus bacillarus, was genetically transformed to express a functional recombinant antibody (Durvasula et al. 2006, incorporated herein by reference in its entirety). S. bacillarus was transformed to produce a murine antibody (rDB3) against progesterone, using a heterologous expression system. In competitive ELISA studies, the rDB3 antibody bound progesterone in a dose-dependent and specific manner. No cross-reactivity with testosterone, a structurally similar steroid, was detected. This study demonstrated that a transgenic cyanobacterium expressed an active recombinant antibody and serves as a model for future applications of this technology.

### Example 3

### Bioamplification of Foreign Gene Products Through Transgenic Micro Algae

An alternate strategy for delivery of transgenic Dunaliella to the target animal is via bioamplification. In this strategy a feed organism such as Artemia initially consumes the transgenic Dunaliella. The engorged Artemia is then fed to the target animal. In this manner, the supplement is bioamplified as it progresses up the food-chain. Artemia are non-selective filter feeders and therefore will ingest a wide range of foods. The main criteria for food selection are particle size, digestibility, and nutrient levels. (Dobbeleir et al. 1980). Possibly the best foods for Artemia are live micro algae such as Nannochloropsis, Tetraselmis, Isochrysis, Dunaliella and Pavlova. Combinations of live phytoplanktons fed to Artemia cultures have demonstrated superior enrichment characteristics over feeding single phytoplankton species (D'Agostino 1980). However, not all species of unicellular algae are appropriate for sustaining Artemia growth. For example, Chlorella and Stichococcus have a thick cell wall that cannot be digested by Artemia.

### Example 4

### Construction of Shuttle Plasmid pRrMDWK6 and Transformation of Synechococcus bacillarus

Electro-competent Synechococcus was generated by adapting protocols for E. coli. The expression-shuttle plasmid, pRrMDWK6, was constructed using a gene encoding a murine three-domain VHK antibody fragment (rDB3) which binds progesterone (He et al., 1995, incorporated herein by reference in its entirety). The expression of this antibody fragment in the Artemia serves as a model system to establish conditions for the eventual expression of functional antibody fragments that will target surface determinants of different viral and bacterial marine pathogens. Binding affinity and specificity of rDB3 closely resemble those of the parent IgG1 antibody; the binding constant is in the order of 1×10⁹ litres/mol. Expression and secretion of rDB3 was under control of a heterologous promoter±signal peptide complex derived from the alpha antigen gene (MKα) of Mycobacterium kansasii (Matsuo et al. 1990). The R. rhodnii replication origin fragment from the shuttle plasmid pR1.1 (Beard et al., 1992) was restricted and cloned into the EcoR1 restriction site of the DNA vector pBluescript SK+ (Stratagene). MKα was amplified using the polymerase chain reaction (PCR) oligonucleotide primers: (SEQ ID NO: 1) 5′-GC TCT AGA GTT AAC TAT TCT TTG TAC GCG-3′ (forward) and (SEQ ID NO: 2) 5′-GC GAA CGC TCC CGC GGT CGC-3′(reverse). The forward primer incorporated a 5′ Xba1 site and the reverse primer contained a native Sac II site. The gene encoding the single-chain antibody fragment DB3VH/K was amplified using the PCR oligonucleotide primers (SEQ ID NO: 3): 5′-GC ACC GCG GGA GCC CAG GTG AAA CTG CTC-3′ (forward) and (SEQ ID NO: 4): 5′-CCT CGA TTGCGG CCG CTT AAC-3′ (reverse). The forward primer included a Sac II site which allowed for ligation in frame with the DB3 fragment and the MKa sequence. The reverse fragment contained a 3′ XbaI site. The ligated MKaDB3VH/K fragment was cloned into the baI site of the shuttle vector. Cloning of a kanamycin resistance gene (Pharmacia) as a Bam HI fragment yielded the final shuttle plasmid pRrMDWK6. Transformation of S. bacillarus with pRrMDWK6 was done as previously described (Durvasula et al 2006, incorporated herein by reference in its entirety).

### Example 5

### Detection of MDWK6 Shuttle Plasmid in Synechococcus sp.

Individual colonies of the transformed Synechococcus were picked and were grown in one liter of F/2 with 20% BHI, G medium additions and kanamycin (501 g/ml). A minimum inhibitory concentration (MIC) was completed by growing transformed and wild-type Synechococcus in increasing concentrations of kanamycin (25, 50, 75, 100, 200, −250 μg kan/ml). The transformed Synechococcus had a MIC of 250 ug/ml in comparison with the non transformed Synechococcus which had a MIC of <50 ug/ml.

PCR was performed on the Synechococcus lysate using primers specific to the kanamycin resistance gene. KANF 5′ (SEQ ID NO: 5): GCTCAGTGGAACGAAAACTCA and KANR5′: (SEQ ID NO: 6) CAATTACAAACAGGAATCGAATG. 5 μl of lysate was used as template. The PCR was performed under the following cycling conditions: 1) a single cycle of 90° C. for 3 minutes followed by 30 cycles of 94° C. for 1 minute, 52° C. for 1 minute, 72° C. for 30 seconds and a single cycle elongation step of 72° C. for 10 minutes. The kanamycin resistant fragment of 500 bp could be amplified only from the transformed Synechococcus.

### Example 6

### Expression and Secretion of a Functional Mouse Specific Progesterone Binding Antibody

Western blot analysis was performed on the untransformed and transformed Synechococcus lysate. An SDS-PAGE gel was run with 100 μg of total protein content. The blot was then transferred to a PVDF membrane (Immobilon, Milipore) and blocked with 5% non-fat milk in TBS (Tris buffered saline containing 1% tween 20) and washed thoroughly with TBS. The blot was then probed with an HRP-linked secondary anti-mouse antibody at a dilution of 1:5000 and developed using an ECL chemiluminescent detection system (Perkin Elmer). Reactive bands were detected using BioMax MR film (Eastman Kodak, Rochester, N.Y., USA) only in the lanes that had the transformed Synechococcus lysate showing that a mouse specific antibody can be expressed by the transformed Synechococcus. ELISA and competitive ELISA were performed on the transformed and untransformed S lysate. Progesterone and testosterone at a concentration of 3 μg/well were coated on micro titer plates. Lysates from transformed and non transformed Synechococcus that were diluted serially were added to the progesterone (BSA conjugate, Sigma) or testosterone (BSA conjugate, Fitzgerald) coated wells and incubated at 16° C. overnight. The secondary antibody used was AP-linked anti-mouse IgG (Chemicon). Color was developed by addition of 4-Nitrophenol Phosphate tablets (Roche) and read at 410 nm. A competitive ELISA using free progesterone was also conducted similar to He, et al. Here, the progesterone was used as an inhibitor at concentrations that would yield 50% of the maximal binding as detected by odometer readings. The mixture contained free progesterone-3-carboxymethyloxime (CMO) and was incubated on progesterone-BSA coated plates. The binding was detected as in the ELISA assay above.

### Example 7

### Establishment of Paratransgenic Artemia spp.

One to two (1-2) Liter of transformed Synechococcus was cultured in Seawater-LB broth containing 50 μg/ml kanamycin. After 2-3 days of growth, it was centrifuged at 5000 rpm for 15 m and the media was drained off. The cells were suspended in sterilized normal saline and centrifuged for another 15 min at 4000 rpm. This step repeated for 4 times to remove the residual media and kanamycin. The final cells were suspended in 10 ml (2×10⁷) normal saline.

Artemia eggs (Brine Shrimp Direct Inc) were allowed to hatch in sterilized sea water. Three days after hatching, the hatchlings were transferred to 20 L glass aquarium with proper aeration. One batch of Artemia was fed with transformed Synechococcus spp. (2×10⁷ cells/ml) and another fed with untransformed Synechococcus (2×10⁷ cells/ml). Feeding was repeated once in 2 days for 6 days. A known volume of Artemia were harvested once in 2 days and were thoroughly washed 8-10 times with sterilized seawater and filtered through a 0.4 micrometer to remove any cynaobaceteria adhered on the surface. This Artemia sample was used for further molecular analysis. Expression and secretion of a functional mouse specific progesterone binding antibody from paratransgenic Artemia was measured by plating a portion of sonicated Artemia lysate in seawater agar containing 50 μl/ml kanamycin to detect the growth of any colonies in the plates. Alternatively we transformed Synechococcus bacillarus with a plasmid CD3-377 (ABRC, Columbus, Ohio) expressing GFP protein. Fluorescence microscopy revealed that protein expressed by cyanobacteria was present in the gut of paratransgenic Artemia fed with S. bacillarus expressing GFP.

### Example 8

### Expression of Peneidin-Like Antimicrobial Peptide (AMP) in Paratransgenic Artemia spp.

Penaeidin-like AMP was cloned and characterized from the hemocytes of Tiger shrimp (Penaeus monodon). The deduced amino acid sequence of this antimicrobial peptide consisted of 55 amino acid residues of the mature peptide and a signal peptide of 19 amino acids with potent antibacterial activity against Vibrio harveyi, Vibrio alginolyticus and Aerococcus viridans (Chiou et al. 2005). We have made de novo synthesized gene for AMP by adjusting the codon bias for optimal cyanobacterial protein expression according to Wilber et al. 1990, incorporated herein by reference in its. A SacII and XbaI restriction site was inserted at the 5′ and 3′ end respectively in the gene sequence to clone into our shuttle plasmid, pRrMDWK6.

### Example 9

### Bacterial Challenge Studies with Vibrio Harveyi in Shrimp Fed Paratransgenic Artemia Spp.

Two routes of feeding of P. monodon nauplii was used in these studies. In one set of studies, transformed cyanobacteria was fed directly via a wet feed preparation. The other route was involved feeding Artemia that have accumulated transgenic cyanobacteria. In either case, we assessed gut expression levels of the recombinant scFv or peptide. P. monodon nauplii was challenged with Vibrio harveyi as per the protocols of Chen et al. (2000), incorporated herein by reference in its entirety. We measured total mortality in the experimental group (carrying genetically transformed Synechococcus) versus the control groups (carrying wild-type Synechococcus or Synechococcus expressing an inert marker antibody reference). Each trial involved 1000 P. monodon nauplii and performed in triplicate. In summary, we tested pathogen specific molecules via 2 different feed strategies in this study. The protection against V harveyi was calculated as the relative percent survival. Statistical analysis of the survival rates among the groups was performed using the chi-square test at a 5% confidence level. The results demonstrated full mortality in the control groups and statistically significant increase in survival in the groups fed transgenic cyanobacteria.

All references discussed herein are specifically incorporated herein by reference. One skilled in the art will readily appreciate that the present invention is well adapted to carry out the objects and obtain the ends and advantages mentioned, as well as those inherent therein. The present invention may be embodied in other specific forms without departing from the spirit or essential attributes thereof and, accordingly, reference should be made to the appended claims, rather than to the foregoing specification, as indicating the scope of the invention.

### REFERENCES

Apt, K. E., P. G. Kroth-Pancic, and A. R. Grossman. 1996. Stable nuclear transformation of the diatom Phaeodactylum tricornutum. Mol Gen Genet. 252:572-9.
Austin, B, E Baudet, and M B C Stobie. 1992. Inhibition of bacterial fish pathogens by Tetraselmis suecica. J of Fish Diseases 15:55-61.
Austin, B, and JG Day. 1990. Inhibition of prawn pathogenic Vibro spp by a commercial spray-dried preparation of Tetraseelmis suecica. Aquaculture 90:389-392.
Avendano, R. E., and C. E. Riquelme. 1999. Establishment of mixed-culture probiotics and microalgae as food for bivalve larvae. Aquaculture Research 30:893-900.
Beard, C. B., C. Cordon-Rosales, and R. V. Durvasula. 2002. Bacterial symbionts of the triatominae and their potential use in control of Chagas disease transmission. Annu Rev Entomol 47:123-41.
Ben-Amotz, A., and M. Avron. 1990. The biotechnology of cultivating the halotolerant alga Dunaliella. Trends Biotechnology 8:121-126.
Ben-Amotz, A., A. Shaish, and M. Avron. 1989. Mode of action of the passively accumulated beta-Carotene of Dunaliella bardawil in protecting the alga against damage by excess irradiation. Plant Physiol 91:10470-1043.
Bextine, B., C. Lauzon, S. Potter, D. Lampe, and T. A. Miller. 2004. Delivery of a genetically marked Alcaligenes sp. to the glassy-winged sharpshooter for use in a paratransgenic control strategy. Curr Microbiol 48:327-31.
Biering, E, S Villoing, I Sommerset, and K E Christie. 2005. Update on viral vaccines for fish. Dev Biol (Base1) 121:97-113.
Boudinot, P., M. Blanco, P. de Kinkelin, and A. Benmansour. 1998. Combined DNA immunization with the glycoprotein gene of viral hemorrhagic septicemia virus and infectious hematopoietic necrosis virus induces double-specific protective immunity and nonspecific response in rainbow trout. Virology 249:297-306.
Boynton, J. E., N. W. Gillham, E. H. Harris, J. P. Hosler, A. M. Johnson, A. R. Jones, B. L. Randolph-Anderson, D. Robertson, T. M. Klein, K.-B. Shark, and et al. 1988. Chloroplast transformation in Chlamydomonas with high velocity microprojectiles. Science 240:1534-8.
Brown, L. E., S. L. Sprecher, and L. R. Keller. 1991. Introduction of exogenous DNA into Chlamydomonas reinhardtii by electroporation. Mol Cell Biol 11:2328-32. Campbell, R., A. Adams, M. F. Tatner, M. Chair, and P. Sorgeloos. 1993. Uptake of Vibrio angillarum vaccine by Artemia salina as a potential oral delivery system to fish fry. Fish Shellfish Immunnol 3:451-459.
Chair, M., M. Dehasque, S. Van Poucke, H. Nelis, P. Sorgeloos, and A. P. De Leenheer. 1994a. An oral challenge for turbot larvae with Vibrio anguillarum. Aquac Int 2:270-272.
Chair, M., R. S. J. Gapasin, M. Dehasque, and P. Sorgeloos. 1994b. Vaccination of European sea bass fry through bioencapsulation of Artemia nauplii. Aquac Int 2:254-261.
Chappel, J. A., W. O. Rogers, S. L. Hoffman, and A. S. Kang. 2004. Molecular dissection of the human antibody response to the structural repeat epitope of Plasmodium falciparum sporozoite from a protected donor. Malar J 3:28.
Chiou, T. T., J. L. Wu, T. T. Chen, and J. K. Lu. 2005. Molecular cloning and characterization of cDNA of penaeidin-like antimicrobial peptide from tiger shrimp (Penaeus monodon). Mar Biotechnol (NY) 7:119-27.
Chou, H. V., C. Y. Huang, C. F. Lo, and G. H. Kou. 1998. Studies on transmission of white spot associated baculovirus (WSBV) in Penaeus monodon and P. japonicus via water borne contact and oral ingestion. Aquaculture 164:263-276.
Clark, T G, and D cassidy-Hanley. 2005. Recombinant subunit vaccines: potentials and constraints. Dev Biol (Base1) 121:151-163. Corbeil, S., S. E. Lapatra, E. D. Anderson, J. Jones, B. Vincent, Y. L. Hsu, and G.
Kurath. 1999. Evaluation of the protective immunogenicity of the N, P, M, NV and G proteins of infectious hematopoietic necrosis virus in rainbow trout oncorhynchus mykiss using DNA vaccines. Dis Aquat Organ 39:29-36.
D'Agostino, A. S. The vital requirements of Artemia: physiology and nutrition. pp. 55-82. In G. Personne, P. Sorgeloos, O. Roels and E. Jaspers. [eds] 1980. The Brine Shrimp Artemia, Wetteren, Balgium, Universa Press.
Debuchy, R., S. Purton, and J. D. Rochaix. 1989. The argininosuccinate lyase gene of Chlamydomonas reinhardtii: an important tool for nuclear transformation and for correlating the genetic and molecular maps of the ARG7 locus. EMBO J. 8:2803-9.
Dhert, P., M. Lavens, M. Duray, and P. Sorgeloos. 1999. Improved larval survival at metamorphosis of Asian seabass (Lates calcarifer) using omega 3-HUFA-enriched live food. Aquaculture 90:63-74.
Dobbeleir, J., N. Adam, E. Bossuyt, E. Bruggeman, and P. Sorgeloos. New asspects of the use of inert diets for high density culturing of brine shrimp. pp. 165-174. In G. Personne, P. Sorgeloos, O. Roels and E. Jaspers. [eds] 1980. The Brine Shrimp Artemia, Wetteren, Belgium, Universa Press.
Dotson, E. M., B. Plikaytis, T. M. Shinnick, R. V. Durvasula, and C. B. Beard. 2003.

Transformation of Rhodococcus rhodnii, a symbiont of the Chagas disease vector Rhodnius prolixus, with integrative elements of the LI mycobacteriophage. Infect Genet Evol 3:103-9.
Dunahay, T. G. 1993. Transformation of Chlamydomonas reinhardtii with silicon carbide whiskers. Biotechniques 15:452-5, 457-8, 460.
Dunahay, T. G., E. E. Jarvis, and P. G. Roessler. 1995 Genetic transformation of the diatoms Cyclotella cryptica and Navicula saprophila. J. Phycol 31:1004-1012.
Durvasula, R. V., A. Gumbs, A. Panackal, O. Kruglov, S. Aksoy, R. B. Merrifield, F. F. Richards, and C. B. Beard. 1997. Prevention of insect-borne disease: An approach using transgenic symbiotic bacteria. Proc Natl Acad Sci USA 94:3274-8.
Durvasula, R. V., A. Gumbs, A. Panackal, O. Kruglov, J. Taneja, A. S. Kang, C. Cordon-Rosales, F. F. Richards, R. G. Whitham, and C. B. Beard. 1999. Expression of a functional antibody fragment in the gut of Rhodnius prolixus via transgenic bacterial symbiont Rhodococcus rhodnii. Med Vet Entomol 13:115-9.
Durvasula, R V, R Sundaram, S Matthews, P Sundaram, and DV Subba Rao. 2006. Prospects for paratransgenic applications to commercial mariculture using genetically engineered algae. pp 864-889, Ch 24 In DV Subba Rao [ed] 2006. Algal Cultures, Analogues of Blooms and Applications, Plymouth, United Kingdom: Science Publishers.
FAO. 2004. The State of World Fisheries and Aquaculture (SOFIA). Rome Food and Agriculture Organization of the United Nations [eds].
Fisher, H, I Robl, M Sumper, and N Kroger. 1999. Targeting and covalent modification of cell wall and membrane proteins heterologously expressed in the diatom Cylindrotheca fusicormis (Bacillariophyceae). J Phycol 35:113-120.
Fisher, M., I. Gokhman, U. Pick, and A. Zamir. 1996. A salt-resistant plasma membrane carbonic anhydrase is induced by salt in Dunaliella salina. J Biol Chem 271:17718-23.
Fisher, M., I. Gokhman, U. Pick, and A. Zamir. 1997. A structurally novel transferrin-like protein accumulates in the plasma membrane of the unicellular green alga Dunaliella salina grown in high salinities. J Biol Chem 272:1565-70.
Geng, D, Y Wang, P Wang, W Li, and Y. Sun. 2003. Stable expression of hepatitis B surface antigen gene in Dunaliella salina. J Applied Phycol 15:451-456.
Gonzalez, Mariela A., Annette W. Coleman, Patricia I. Gomez, and Rolando Montoya. 2001. Phylogenetic Relationship among various strains of Dunaliella (Chlorophyceae) based on nuclear 18S rDNA sequences J Phycol 37:604-611.
Grisez, L., M. Chair, P. Sorgeloos, and F. Ollevier. 1996. Mode of infection and spread of Vibrio anguillarum in turbot Scophthalmus maximus larvae after oral challenge through live feed. Dis. Aquat Org 26:181-187.
Gumpel, N. J., and S. Purton. 1994. Playing tag with Chlamydomonas. Trends Cell Biol 4:299-301.
Hallmann, A., and M. Sumper. 1994. Reporter genes and highly regulated promoters as tools for transformation experiments in Volvox carteri. Proc Natl Acad Sci USA 91:11562-6.
Hallmann, A, and M. Sumper. 1996. The Chlorella hexose/H+ symporter is a useful selectable marker and biochemical reagent when expressed in Volvox. Proc Natl Acad Sci USA 93:669-73.
Hansen, E., K. Fernandes, G. Goldspink, P. Butterworth, P. K. Umeda, and K. C. Chang. 1991. Strong expression of foreign genes following direct injection into fish muscle. FEBS Lett 290:73-6.
Heppel, J, and H L Davis. 2000. Application of DNA vaccine technology to aquaculture. Adv Drug Delivery Rev 43:29-43.
Heppel, J, N Lorenzen, N K Armstrong, T Wu, E Lorenzen, and K Einer-Jensen. 1998. Development of DNA vaccines for fish:Vector design, intramuscular injection and antigen expression using viral haemorrhagic septicimia virus genes as model. Fish and Shellfish Immunology 8:271-286.
Hill, B J. The need for effective disease control in international aquaculture. pp. 3-12. In P. J. Midtlyng. [eds] 2005. Progress in Fish Vaccinology, Bergen, Norway. Karger.
Irianto, A, and B Austin. 2002. Use of probiotics to control furunculosis in Rainbow trout, Oncorhynchus mykiss. J Fish Diseases 25:1-10.
Jiang, G. Z., Y. M. Lu, X. L. Niu, and L. X. Xue. 2005. The actin gene promoter-driven bar as a dominant selectable marker for nuclear transformation of Dunaliella salina. Yi Chuan Xue Bao 32:424-33.
Joosten, P. H. M., M. Aviles-Trigueros, P. Sorgeloos, and J. H. W. M. Rombout. 1995. Oral vaccination of the juvenille carp (Cyprinus carpio) and gilthead seabream (Sparus aurata) with bioencapsulated Vibrio anguillarum bacterin. Fish Shellfish Immunnol 5:289-299.
Kanellos, T., I. D. Sylvester, C. R. Howard, and P. H. Russell. 1999. DNA is as effective as protein at inducing antibody in fish. Vaccine 17:965-72.
Kim, C. H., M. C. Johnson, J. D. Drennan, B. E. Simon, E. Thomann, and J. A. Leong. 2000. DNA vaccines encoding viral glycoproteins induce nonspecific immunity and Mx protein synthesis in fish. J Virol 74:7048-54.
Kurath, G., K. A. Garver, S. Corbeil, D. G. Elliott, E. D. Anderson, and S. E. LaPatra. 2006. Protective immunity and lack of histopathological damage two years after DNA vaccination against infectious hematopoietic necrosis virus in trout. Vaccine 24:345-54.
Li, H. X., X. L. Meng, J. P. Xu, W. Lu, and J. Wang 2005. Protection of crayfish, Cambarus clarkii, from white spot syndrome virus by polyclonal antibodies against a viral envelope fusion protein. J Fish Dis 28:285-91.
Lin, J H, C C Yu, C C Lin, and H L Yang. 2005. An oral delivery system for recombinant subunit vaccine to fish. Dev Biol (Base1) 121:175-180.
Lorenzen, N., E. Lorenzen, and K. Einer-Jensen. 2001. Immunity to viral haemorrhagic septicaemia (VHS) following DNA vaccination of rainbow trout at an early life-stage. Fish Shellfish Immunol 11:585-91.
Lorenzen, N., E. Lorenzen, K. Einer-Jensen, J. Heppell, and H. L. Davis. 1999. Genetic vaccination of rainbow trout against viral haemorrhagic septicaemia virus: small amounts of plasmid DNA protect against a heterologous serotype. Virus Res 63:19-25.
Lorenzen, N., E. Lorenzen, K. Einer-Jensen, and S. E. LaPatra. 2002. Immunity induced shortly after DNA vaccination of rainbow trout against rhabdoviruses protects against heterologous virus but not against bacterial pathogens. Dev Comp Immunol 26:173-9.
Luo, T., X. Zhang, Z. Shao, and X. Xu. 2003. PmAV, a novel gene involved in virus resistance of shrimp Penaeus monodon. FEBS Letters 551:53-57.
Maul, J. E., J. W. Lilly, L. Cui, C. W. dePamphilis, W. Miller, E. H. Harris, and D. B. Stem. 2002. The Chlamydomonas reinhardtii plastid chromosome: islands of genes in a sea of repeats. Plant Cell 14:2659-79.
Mayfield, S. P., S. E. Franklin, and R. A. Lerner. 2003. Expression and assembly of a fully active antibody in algae. Proc Natl Acad Sci USA 100:438-42. Nusbaum, K. E., B. F. Smith, P. Delnnocentes, and R. C. Bird. 2002. Protective immunity induced by DNA vaccination of channel catfish with early and late transcripts of the channel catfish herpes virus (IHV-1). Vet Immunol Immunopathol 84:151-68.
Otoshi, C. A., S. M. Arce, and S. M. Moss. 2002. Use of recirculating systems for the production of broodstock shrimp. Panorama Acuicola Magazine 8.
Otoshi, C A, A D Montgomery, A M Look, and S M Moss. 2001. Effects of diet and water source on the nursery production of Pacific white shrimp Litopenaeus vannamei. J World Aquaculture Society 32:243-249.
Platt, T., DV Subba Rao. and B Irwin. 1983. Photosynthesis of Picoplankton in the oligotrophic ocean Nature, Lond.301: 702-704.
Rabbani, S., P. Beyer, J. Lintig, P. Hugueney, and H. Kleinig. 1998. Induced beta-carotene synthesis driven by triacylglycerol deposition in the unicellular alga Dunaliella bardawil. Plant Physiol 116:1239-48.
Raengpipat, S, W Phianphak, S Piyatiratitivorakul, and P Menasveta. 1998. Effects of probiotic bacterium in black tiger shrimp Penaeus monodon survival and growth. Aquaculture 167:301-313.
Rahman, A., and N. Maclean. 1992. Fish transgene expression by direct injection into fish muscle. Mol Mar Biol Biotechnol 1:286-9.
Robalino, J., T. Bartlett, E. Shepard, S. Prior, G. Jaramillo, E. Scura, R. W. Chapman, P. S. Gross, C. L. Browdy, and G. W. Warr. 2005. Double-stranded RNA induces sequence-specific antiviral silencing in addition to nonspecific immunity in a marine shrimp: convergence of RNA interference and innate immunity in the invertebrate antiviral response? J Virol 79:13561-71.
Robalino, J., C. L. Browdy, S. Prior, A. Metz, P. Parnell, P. Gross, and G. Warr. 2004. Induction of antiviral immunity by double-stranded RNA in a marine invertebrate. Virol 78:10442-8.
Rosenberry, B. 2005. About Shrimp Farming. ShrimpNews.
Sanchez-Estudillo, L., Y. Freile-Pelegrin, R. Rivera-Madrid, D. Robledo, and J. A. Narvaez-Zapata. 2006. Regulation of two photosynthetic pigment-related genes during stress-induced pigment formation in the green alga, Dunaliella salina. Biotechnol Lett 28:787-91.
Schiedlmeier, B., R. Schmitt, W. Muller, M. M. Kirk, H. Gruber, W. Mages, and D. L. Kirk. 1994. Nuclear transformation of Volvox carteri. Proc Natl Acad Sci USA 91:5080-4.
Scholz, U, G Garcia Diaz, D Ricque, L E Cruz Suarez, F Vargas Albores, and J. Latchford. 1999. Enhancement of vibriosis resistance in juvenile Penaeus vannamei by supplementation of diets with different yeast products. Aquaculture 176:271-283.
Shimogawara, K., S. Fujiwara, A. Grossman, and H. Usuda. 1998. High-efficiency transformation of Chlamydomonas reinhardtii by electroporation. Genetics 148:1821-8.
Sun, X. M., Y. P. Tang, X. Z. Meng, W. W. Zhang, S. Li, Z. R. Deng, Z. K. Xu, and R. T. Song. 2006a. Sequencing and analysis of a genomic fragment provide an insight into the Dunaliella viridis genomic sequence. Acta Biochim Biophys Sin (Shanghai) 38:812-20.
Sun, Y., X. Gao, Q. Li, Q. Zhang, and Z. Xu. 2006b. Functional complementation of a nitrate reductase defective mutant of a green alga Dunaliella viridis by introducing the nitrate reductase gene. Gene 377:140-9.
Sundaram, R. K., I. Hurwitz, S. Matthews, E. Hoy, S. Kurapati, C. Crawford, P. Sundaram, and R. V. Durvasula. 2006. Expression of a Functional Single Chain Antibody in Corynebacterium pseudodiphtheriticum. Appl. Env. Microbiol. (in review).
Takano, T., A. Iwahori, I. Hirono, and T. Aoki. 2004. Development of a DNA vaccine against hirame rhabdovirus and analysis of the expression of immune-related genes after vaccination. Fish Shellfish Immunol 17:367-74. Tirasophon, W., Y. Roshorm, and S. Panyim. 2005. Silencing of yellow head virus replication in penaeid shrimp cells by dsRNA. Biochem Biophys Res Commun 334:102-7.
Vahrenholz, C., G. Riemen, E. Pratje, B. Dujon, and G. Michaelis. 1993. Mitochondrial DNA of Chlamydomonas reinhardtii: the structure of the ends of the linear 15.8-kb genome suggests mechanisms for DNA replication. Curr Genet. 24:241-7.
van Hulten, M. C., J. Witteveldt, M. Snippe, and J. M. Vlak. 2001. White spot syndrome virus envelope protein VP28 is involved in the systemic infection of shrimp. Virology 285:228-33.
Vaseeharan, B., T. Prem Anand, T. Murugan, and J. C. Chen. 2006. Shrimp vaccination trials with the VP292 protein of white spot syndrome virus. Lett Appl Microbiol 43:137-42.
Venegas, C. A., L. Nonaka, K. Mushiake, T. Nishizawa, and K. Murog. 2000. Quasi-immune response of Penaeus japonicus to penaeid rod-shaped DNA virus (PRDV). Dis Aquat Organ 42:83-9.
Verri, T., L. Ingrosso, R. Chiloiro, A. Danieli, V. Zonno, P. Alifano, N. Romano, G. Scapigliati, S. Vilella, and C. Storelli. 2003. Assessment of DNA vaccine potential for gilthead sea bream (Sparus aurata) by intramuscular injection of a reporter gene. Fish Shellfish Immunol 15:283-95.
Verschuere, L., G. Rombaut, P. Sorgeloos, and W. Verstraete. 2000. Probiotic bacteria as biological control agents in aquaculture. Microbiol. Mol Biol Rev 64:655-71.
Walker, T. L., D. K. Becker, and C. Collet. 2005. Characterisation of the Dunaliella tertiolecta RbcS genes and their promoter activity in Chlamydomonas reinhardtii. Plant Cell Rep 23:727-35.
Witteveldt, J., C. C. Cifuentes, J. M. Vlak, and M. C. van Hulten. 2004. Protection of Penaeus monodon against white spot syndrome virus by oral vaccination. J Virol 78:2057-61.
Witteveldt, J., J. M. Vlak, and M. C. van Hulten. 2006. Increased tolerance of Litopenaeus vannamei to white spot syndrome virus (WSSV) infection after oral application of the viral envelope protein VP28. Dis Aquat Organ 70:167-70.
Wu, J. L., T. Nishioka, K. Mori, T. Nishizawa, and K. Muroga. 2002. A time-course study on the resistance of Penaeus japonicus induced by artificial infection with white spot syndrome virus. Fish Shellfish Immunol 13:391-403.
Zaslavskaia, L. A., J. C. Lippmeier, C. Shih, D. Ehrhardt, A. R. Grossman, and K. E. Apt. 2001. Trophic conversion of an obligate photoautotrophic organism through metabolic engineering. Science 292:2073-5.

What is claimed is:

1. A paratransgenic method for prevention, amelioration or treatment of a disease or disorder in an aquatic animal comprising: i) providing a genetically modified micro algae that expresses one or more recombinant molecules that specifically target one or more key epitopes of a pathogen that infects the aquatic animal and ii) feeding the aquatic animal directly or indirectly with the genetically modified micro algae.

2. The paratransgeneic method of claim 1, wherein the micro algae comprises Dunaliella or a variant thereof.

3. The paratransgeneic method of claim 1, wherein the recombinant molecule comprises one or more immunogenic peptides, single chain antibody fragments, DNA vaccine, or a combination thereof.

4. The paratransgeneic method of claim 3, wherein the single chain antibody fragment specifically binds to one or more key epitopes of a pathogen.

5. The paratransgeneic method of claim 4, wherein the pathogen comprises a virus, bacterium, protozoa, or a combination thereof.

6. The paratransgeneic method of claim 5, wherein the single chain antibody fragment blocks assembly of the virus by inhibiting expression of one or more viral proteins.

7. The paratransgeneic method of claim 5, wherein the virus comprises White Spot Syndrome Virus (WSSV), or variants and serotypes thereof.

8. The paratransgeneic method of claim 1, wherein the recombinant molecule comprises one or more antibacterial molecules.

9. The paratransgeneic method of claim 8, wherein the antibacterial molecules comprise Peneidin-Like antimicrobial peptide AMP.

10. The paratransgeneic method of claim 1, wherein the genetically modified micro algae is bioamplied in a probiotic organism prior to consumption by the aquatic animal.

11. The paratransgeneic method of claim 3, wherein the probiotic organism comprises bacterium, and planktonic organism comprising Artemia, rotifers, copepods, or daphnia, or a combination thereof.

12. The paratransgeneic method of claim 1, wherein the genetically modified micro algae is produced by transformation of a symbiotic or commensal bacteria of the micro algae with a desired genetic material.

13. The paratransgeneic method of claim 1, wherein the micro algae comprises a unicellular micro algae.

14. The paratransgeneic method of claim 13, wherein the micro algae comprises Isochrysis, Pavlova, Nannochloropsis, Thalassiosira psuedonana, Cyanobacterium, Dunaliella, Phaeodactylum tricornutum, Red alga Porphydium cruentum, Haematococcus, Botryococcus, Gymnodinium sp, Gonyaulax, Chlamydomonas, Chlorella pyrenoidosa, or species and variants thereof.

15. The paratransgeneic method of claim 14, wherein the cyanobacterium comprises Cyanobacterium Spirulina, Cyanobacteria Scytonema, cyanobacteria Oscillatoria, or Synechococcus bacillarus, or species and variants thereof.

16. The paratransgeneic method of claim 1, wherein the aquatic animal is a farm-raised animal.

17. The paratransgeneic method of claim 16, wherein the aquatic animal comprises shrimp.

18. The paratransgeneic method of claim 1, wherein the genetically modified micro algae expresses a protein, a peptide, or one or more antibody fragments that inhibit the growth or replication of a pathogen comprising Vibrio species, Taura, and White spot virus.

19. The paratransgeneic method of claim 1, wherein the antibody fragment is a scFv fragment that provides immunity against infections by Vibrio harveyi, White Spot Syndrome Virus, or both.

20. A paratransgeneic method for control of infection in aquaculture comprising: i) providing a genetically modified cynobacteria that expresses a recombinant molecule that specifically targets one or more key peptides of a pathogen that infects an aquatic animal and ii) feeding the aquatic animal directly or indirectly with the genetically modified cynobacteria.


SEQUENCE LISTING (6 sequences)

SEQ ID NO: 1 - DNA, 29 bp, Artificial Sequence
  Primer
gctctagagttaactattctttgtacgcg

SEQ ID NO: 2 - DNA, 20 bp, Artificial Sequence
  Primer
gcgaacgctcccgcggtcgc

SEQ ID NO: 3 - DNA, 29 bp, Artificial Sequence
  Primer
gcaccgcgggagcccaggtgaaactgctc

SEQ ID NO: 4 - DNA, 21 bp, Artificial Sequence
  Primer
cctcgattgcggccgcttaac

SEQ ID NO: 5 - DNA, 21 bp, Artificial Sequence
  Primer
gctcagtggaacgaaaactca

SEQ ID NO: 6 - DNA, 23 bp, Artificial Sequence
  Primer
caattacaaacaggaatcgaatg